SEQUENCE LISTING (44 sequences)

SEQ ID NO: 1 - DNA, 22 bp, synthetic construct
  source 1..22: mol_type = other DNA
gccaatggtaaacatgacaggc

SEQ ID NO: 2 - DNA, 23 bp, synthetic construct
  source 1..23: mol_type = other DNA
tgtctaggttcacgtacacatct

SEQ ID NO: 3 - DNA, 20 bp, synthetic construct
  source 1..20: mol_type = other DNA
cggggtactcctggtcctac

SEQ ID NO: 4 - DNA, 20 bp, synthetic construct
  source 1..20: mol_type = other DNA
gcatccctacttcccccttg

SEQ ID NO: 5 - DNA, 20 bp, synthetic construct
  source 1..20: mol_type = other DNA
cttcgccgctactcctgttc

SEQ ID NO: 6 - DNA, 22 bp, synthetic construct
  source 1..22: mol_type = other DNA
ccctgagggcaaattgtgaaaa

SEQ ID NO: 7 - DNA, 21 bp, synthetic construct
  source 1..21: mol_type = other DNA
tcagtggcggacttcagaaag

SEQ ID NO: 8 - DNA, 21 bp, synthetic construct
  source 1..21: mol_type = other DNA
gcaaaaaggtgctcgttcttc

SEQ ID NO: 9 - DNA, 22 bp, synthetic construct
  source 1..22: mol_type = other DNA
gaaatgccaccttttgacagtg

SEQ ID NO: 10 - DNA, 21 bp, synthetic construct
  source 1..21: mol_type = other DNA
tggatgctctcatcaggacag

SEQ ID NO: 11 - DNA, 20 bp, synthetic construct
  source 1..20: mol_type = other DNA
agttgccttcttgggactga

SEQ ID NO: 12 - DNA, 20 bp, synthetic construct
  source 1..20: mol_type = other DNA
tccacgatttcccagagaac

SEQ ID NO: 13 - DNA, 19 bp, synthetic construct
  source 1..19: mol_type = other DNA
cctgtagcccacgtcgtag

SEQ ID NO: 14 - DNA, 22 bp, synthetic construct
  source 1..22: mol_type = other DNA
gggagtagacaaggtacaaccc

SEQ ID NO: 15 - DNA, 21 bp, synthetic construct
  source 1..21: mol_type = other DNA
cctgtccatcttagggttgcc

SEQ ID NO: 16 - DNA, 20 bp, synthetic construct
  source 1..20: mol_type = other DNA
ggccttgtaggggtaggaga

SEQ ID NO: 17 - DNA, 20 bp, synthetic construct
  source 1..20: mol_type = other DNA
ggctgtattcccctccatcg

SEQ ID NO: 18 - DNA, 22 bp, synthetic construct
  source 1..22: mol_type = other DNA
ccagttggtaacaatgccatgt

SEQ ID NO: 19 - DNA, 20 bp, synthetic construct
  source 1..20: mol_type = other DNA
ttcggtaccggaagctgttg

SEQ ID NO: 20 - DNA, 20 bp, synthetic construct
  source 1..20: mol_type = other DNA
ctggactggggaagtcaacc

SEQ ID NO: 21 - DNA, 20 bp, synthetic construct
  source 1..20: mol_type = other DNA
gtgctgccccagatgattct

SEQ ID NO: 22 - DNA, 20 bp, synthetic construct
  source 1..20: mol_type = other DNA
ggcgctgtcttgagacctaa

SEQ ID NO: 23 - DNA, 20 bp, synthetic construct
  source 1..20: mol_type = other DNA
cggggtgtgcctggtcctac

SEQ ID NO: 24 - DNA, 20 bp, synthetic construct
  source 1..20: mol_type = other DNA
gcatccctacttcccccttg

SEQ ID NO: 25 - DNA, 21 bp, synthetic construct
  source 1..21: mol_type = other DNA
tcagtggaggacttcagaaag

SEQ ID NO: 26 - DNA, 21 bp, synthetic construct
  source 1..21: mol_type = other DNA
gcaaaaaggtgctcgttcttc

SEQ ID NO: 27 - DNA, 23 bp, synthetic construct
  source 1..23: mol_type = other DNA
gacaggattgacagattgatagc

SEQ ID NO: 28 - DNA, 21 bp, synthetic construct
  source 1..21: mol_type = other DNA
agtctcgttcgttatcggaat

SEQ ID NO: 29 - DNA, 20 bp, synthetic construct
  source 1..20: mol_type = other DNA
ggccgtcttcccctccatcg

SEQ ID NO: 30 - DNA, 22 bp, synthetic construct
  source 1..22: mol_type = other DNA
ccagttggtgacaatgccgtgt

SEQ ID NO: 31 - DNA, 21 bp, synthetic construct
  source 1..21: mol_type = other DNA
ggaggggacattactacggtc

SEQ ID NO: 32 - DNA, 21 bp, synthetic construct
  source 1..21: mol_type = other DNA
attccgccgtgaaagtgaaga

SEQ ID NO: 33 - DNA, 20 bp, synthetic construct
  source 1..20: mol_type = other DNA
gtggccgtcagcatagatgg

SEQ ID NO: 34 - DNA, 23 bp, synthetic construct
  source 1..23: mol_type = other DNA
tgaatgtctccctcgaaagtctt

SEQ ID NO: 35 - DNA, 19 bp, synthetic construct
  source 1..19: mol_type = other DNA
accctcaaaccaccacacg

SEQ ID NO: 36 - DNA, 22 bp, synthetic construct
  source 1..22: mol_type = other DNA
gctcaccgtaatcagtcctcta

SEQ ID NO: 37 - DNA, 21 bp, synthetic construct
  source 1..21: mol_type = other DNA
caacgcagacctgatggattt

SEQ ID NO: 38 - DNA, 19 bp, synthetic construct
  source 1..19: mol_type = other DNA
agcccccgcttcttcattc

SEQ ID NO: 39 - DNA, 20 bp, synthetic construct
  source 1..20: mol_type = other DNA
tggaaacgtccgggttacag

SEQ ID NO: 40 - DNA, 20 bp, synthetic construct
  source 1..20: mol_type = other DNA
ctgatccggtccatcttcct

SEQ ID NO: 41 - DNA, 21 bp, synthetic construct
  source 1..21: mol_type = other DNA
catgtacgttgctatccaggc

SEQ ID NO: 42 - DNA, 21 bp, synthetic construct
  source 1..21: mol_type = other DNA
ctccttaatgtcacgcacgat

SEQ ID NO: 43 - protein, 656 aa, synthetic construct
MIFLYQVVHFILFTSVSGECVTQLLKDTCFEGGDITTVFTPSAKYCQVVCTYHPRCLLFTFTAESPSEDPTRWFTCVLKDSVTETLPRVNRTAAISGYSFKQCSHQISACNKDIYVDLDMKGINYNSSVAKSAQECQERCTDDVHCHFFTYATRQFPSLEHRNICLLKHTQTGTPTRITKLDKVVSGFSLKSCALSNLACIRDIFPNTVFADSNIDSVMAPDAFVCGRICTHHPGCLFFTFFSQEWPKESQRNLCLLKTSESGLPSTRIKKSKALSGFSLQSCRHSIPVFCHSSFYHDTDFLGEELDIVAAKSHEACQKLCTNAVRCQFFTYTPAQASCNEGKGKCYLKLSSNGSPTKILHGRGGISGYTLRLCKMDNECTTKIKPRIVGGTASVRGEWPWQVTLHTTSPTQRHLCGGSIIGNQWILTAAHCFYGVESPKILRVYSGILNQSEIKEDTSFFGVQEIIIHDQYKMAESGYDIALLKLETTVNYTDSQRPICLPSKGDRNVIYTDCWVTGWGYRKLRDKIQNTLQKAKIPLVTNEECQKRYRGHKITHKMICAGYREGGKDACKGDSGGPLSCKHNEVWHLVGITSWGEGCAQRERPGVYTNVVEYVDWILEKTQAVTRTRPLEQKLISEEDLAANDILDYKDDDDKV

SEQ ID NO: 44 - protein, 624 aa, synthetic construct
MTSLHQVLYFIFFASVSSECVTKVFKDISFQGGDLSTVFTPSATYCRLVCTHHPRCLLFTFMAESSSDDPTKWFACILKDSVTEILPMVNMTGAISGYSFKQCPQQLSTCSKDVYVNLDMKGMNYNSSVVKNARECQERCTDDAHCQFFTYATGYFPSVDHRKMCLLKYTRTGTPTTITKLNGVVSGFSLKSCGLSNLACIRDIFPNTVLADLNIDSVVAPDAFVCRRICTHHPTCLFFTFFSQAWPKESQRHLCLLKTSESGLPSTRITKSHALSGFSLQHCRHSVPVFCHPSFYNDTDFLGEELDIVDVKGQETCQKTCTNNARCQFFTYYPSHRLCNERNRRGRCYLKLSSNGSPTRILHGRGGISGYSLRLCKMDNVCTTKINPRVVGGAASVHGEWPWQVTLHISQGHLCGGSIIGNQWILTAAHCFSGIETPKKLRVYGGIVNQSEINEGTAFFRVQEMIIHDQYTTAESGYDIALLKLESAMNYTDFQRPICLPSKGDRNAVHTECWVTGWGYTALRGEVQSTLQKAKVPLVSNEECQTRYRRHKITNKMICAGYKEGGKDTCKGDSGGPLSCKYNGVWHLVGITSWGEGCGQKERPGVYTNVAKYVDWILEKTQTV

(19) United States
(12) Patent Application Publication
(10) Pub. No.: US 2025/0345404 A1
(43) Pub. Date: Nov. 13, 2025

(54) COMPOSITIONS AND METHODS FOR TREATING HEART FAILURE

(71) Applicant: The Regents of the University of California, Oakland, CA (US)

(72) Inventors: Aldons Lusis, Los Angeles, CA (US); Yang Cao, Los Angeles, CA (US)

(21) Appl. No.: 18/730,984

(22) PCT Filed: Jan. 27, 2023
(86) PCT No.: PCT/US23/61416

§ 371 Date: Jul. 22, 2024

Related U.S. Application Data
(60) Provisional application No. 63/305,064, filed on Jan. 31, 2022.

(51) Int. Cl. A61K 38/48; A61B 5/00; A61K 35/76; A61K 45/06; A61K 48/00; A61P 9/04; C12N 15/86

(52) U.S. Cl. CPC A61K 38/4846; A61B 5/4848; A61K 35/76; A61K 45/06; A61K 48/0058; A61P 9/04; C12N 15/86; C12Y 304/21027

(57) ABSTRACT

Compositions and methods are provided for treating, preventing, and/or alleviating at least one symptom of heart failure with preserved ejection fraction.

### CROSS-REFERENCE TO RELATED APPLICATIONS

This application is the 35 U.S.C. 371 national phase application of International Patent Application No. PCT/US2023/061416, filed Jan. 27, 2023, which claims the benefit of priority to U.S. Provisional Application No. 63/305,064, filed on Jan. 31, 2022, the contents of each of which are hereby incorporated by reference in their entirety.

### STATEMENT OF GOVERNMENT SUPPORT

This invention was made with government support under Grant No. DK117850 awarded by the National Institutes of Health. The government has certain rights in the invention.

### REFERENCE TO A SEQUENCE LISTING XML

This application contains a Sequence Listing which has been submitted electronically in XML format. The Sequence Listing XML is incorporated herein by reference. Said XML file, created on Feb. 5, 2025, is named UCH-31801_SL.xml and is 41,024 bytes in size.

### BACKGROUND

Heart failure with preserved ejection fraction (HFpEF) is an increasingly prevalent syndrome characterized by diastolic dysfunction and preserved ejection fraction and is distinct from heart failure with reduced ejection fraction (HFrEF) in terms of pathogenesis and effective therapeutic management.⁹ HFpEF, previously referred to as diastolic heart failure, can be characterized by disruptions or dysfunctions in one or more of the following: ventricular diastolic function, left ventricular systolic reserve, systemic and pulmonary vascular function, nitric oxide bioavailability, chronotropic reserve, right heart function, autonomic tone, and left atrial function, as well as peripheral impairments (Borlaug et al., Nature Reviews Cardiology 11: 507-15 (2014)). The complex pathophysiology of HfpEF has hampered efforts to find a therapeutic approach, and current treatment strategies generally limited to controlling volume status and comorbidities (Anderson et al., Current Cardiology Reports 16, Article number: 501 (2014)). HFpEF accounts for half of all cases of heart failure with multiple comorbidities such as diabetes, hypertension, and restrictive cardiomyopathies.^{10,11} For example, chronic systemic inflammation and metabolic disorders affect the myocardium in patients suffering from HFpEF. Since HFpEF is distinct from HFrEF in terms of pathophysiology, effective therapies for HFrEF are largely ineffective for HFpEF. To date, there are no effective therapies for HFpEF.

### SUMMARY OF THE INVENTION

The present invention is based on the discovery that FXI expressed in the liver can ameliorate heart failure with preserved ejection fraction. Accordingly, the present invention is directed to compositions and methods for treating heart failure in a subject. More specifically, compositions and methods presented are for treating heart failure with preserved ejection fraction (HFpEF) by administering an FXI polypeptide or a nucleic acid molecule encoding an FXI polypeptide.

### BRIEF DESCRIPTION OF THE DRAWINGS

FIGS. 1A-1O illustrate a systems genetics approach that identified liver-heart crosstalk is involved the development of HFpEF in C57BL/6J male mice. FIG. 1A is a schematic illustrating the identification of liver-heart interaction using 100 inbred strains of mice from the Hybrid Mouse Diversity Panel (HMDP). The correlation between the secreted factors (from the liver) and cardiac gene expression (RNA-seq) is used for liver-heart predictions. This framework identified peptides that were secreted by the liver and strongly associated with the cardiac transcriptome. N=4-20 for each strain. Also shown is the distribution of significance scores for all liver genes across all heart gene expression in 100 mouse strains and a list of the top 20 genes potentially mediating liver-heart communication. FIG. 1B is an image of a Western blot showing GFP protein in indicated tissues after injection of AAV8-GFP. FIG. 1C is an experimental timeline for the development of HFpEF in C57BL/6J male mice. Mice were injected with AAV8 containing cDNA sequence for GFP or candidate genes, then fed on HFD+1-NAME diet for 7 weeks to induce HFpEF phenotypes. Echo=echocardiography. FIGS. 1D-1O are graphs showing measured characteristics of C57BL/6J male mice that were fed with chow diet or HFD+1-NAME diet for 7 weeks. FIG. 1D shows the E/A ratio. FIG. 1E shows the E/e′ ratio. FIG. 1F shows the heart weight/tibia length ratio. FIG. 1G shows the weight/dry lung weight ratio. FIG. 1H shows LVEF. FIG. 1I shows body weight. FIG. 1J shows fat mass. FIG. 1K shows glucose tolerance test and area under curve. FIG. 1L shows plasma glucose. FIG. 1M shows total cholesterol. FIG. 1N shows unesterified cholesterol. FIG. 1O shows running distance in the C57BL/6J male mice. Each point represents a mouse. All data are presented as the mean±SEM. ns, not significant, *P<0.05, **P<0.01, and ***P<0.001, by Student's t test.

FIGS. 2A-2F show expression data and predicted and functional roles of FXI. FIG. 2A is a graph showing a qRT-PCR analysis of F11 expression across indicated tissues in C57BL/6J mice. N=4. All data are presented as the mean±SEM. FIG. 2B is graph showing a F11 expression across tissues from The Human Protein Atlas (www.proteinatlas.org/ENSG00000088926-F11/tissue). FIG. 2C is an image of a Western blot analysis of FXI protein levels across indicated tissues in C57BL/6J mice. FIG. 2D shows liver F11 expression correlation with clinical traits within the HMDP. FIG. 2E is a table showing pathway enrichment derived from heart genes correlated with liver F11. FIG. 2F is a table showing significant GWAS (genome-wide association studies) loci for indicated clinical traits in human population. GWAS catalog and PhenoScanner databases consist human genotype-phenotype associations from publicly available genetic association studies.

FIGS. 3A-3Z show that FXI overexpression reverses HFpEF-induced diastolic dysfunction, inflammation and fibrosis. FIG. 3A shows plasma FXI levels in C57BL/6J male mice subjected to chow diet and 7 weeks of HFD+1-NAME diet. N=8. FIG. 3B shows an association between plasma FXI levels and the diastolic dysfunction (E/e′ ratio) in C57BL/6J male mice injected with AAV8-GFP or AAV8-F11 and fed with HFD+1-NAME diet for 7 weeks. FIG. 3C shows that plasma FXI levels were inversely correlated with E/e′ ratio in 30 inbred strains of male mice that were fed a +1-NAME diet to induce HFpEF. FIG. 3D and FIG. 3E show an image of a Western blot and a graph of plasma FXI levels, respectively, of samples obtained from C57BL/6J male mice injected with AAV8 containing cDNA sequence for GFP or F11, then fed with HFD+1-NAME diet for 7 weeks. FIG. 3F shows the liver FXI expression in the two groups of mice. FIG. 3G and FIG. 3H show plasma FXI levels detected by Western blot and ponceau S staining, and FIG. 3I, FIG. 3J, and FIG. 3K show the body weight, fat mass, and lean mass, respectively, in the two groups of mice. FIG. 3L shows the E/A ratio. FIG. 3M shows the E/e′ ratio. FIG. 3N comprises representative images of echocardiography results. FIG. 3O shows left ventricle ejection fraction (LVEF) in the two groups of mice. FIG. 3P shows the heart weight/tibia length ratio and FIG. 3Q shows the lung weight (wet/dry ratio) of the two groups of mice. FIG. 3R is representative echocardiogram images of the two groups of mice. FIG. 3S shows the running distance achieved by these mice. FIG. 3T, FIG. 3U, FIG. 3V, and FIG. 3W are graphs of the white (gonadal fat) and brown adipose weight measured at sacrifice, plasma total cholesterol (TC), unesterified cholesterol (UC), and free fatty acids (FFA), respectively, observed in the two groups of mice. FIG. 3X, FIG. 3Y, and FIG. 3Z are graphs showing the results of a glucose tolerance test and area under curve, plasma glucose, and plasma insulin, respectively, observed in the two groups of mice. For FIGS. 3A, 3E, and 3L-3S, all data are presented as the mean±SEM. ns, not significant. *P<0.05, **P<0.01, ***P<0.001, and ****p<0.0001, by Student's t test (FIGS. 3A, 3E, 3T, and 3X-3Z) or by 2-way ANOVA (FIGS. 3L-3S and 3U-3W). Each point represents a mouse. All data are presented as the mean±SEM. ns, not significant. *P<0.05, **P<0.01, ***P<0.001, and ****p<0.0001, by 2-way ANOVA.

FIGS. 4A-4B show the effects of FXI overexpression on blood coagulation. FIG. 4A and FIG. 4B show thrombin-antithrombin complexes (TAT) and mean platelet volume (MPV), respectively, in C57BL/6J male mice injected with AAV8 containing cDNA sequence for GFP or F11, then fed with HFD+1-NAME diet for 7 weeks. For these figures, each point represents a mouse. All data are presented as the mean±SEM. ns, not significant. *P<0.05, **P<0.01, ***P<0.001, and ****p<0.0001 by Student's t test (FIG. 4A) or by analyzed by 2-way ANOVA (FIG. 4B).

FIGS. 5A-5I show the effects of FXI on cardiac infiltration of inflammatory cells and fibrosis. FIG. 5A and FIG. 5B show number of blood cells and cytokines, respectively, in C57BL/6J male mice injected with AAV8 containing cDNA sequence for GFP or F11, then fed with HFD+1-NAME diet for 7 weeks. FIG. 5C shows relative mRNA levels of indicated genes in the hearts of these two groups of mice (N=8-10). FIG. 5D is a qRT-PCR analysis of indicated genes in C57BL/6J male mice fed a chow diet or HFD+1-NAME diet for 7 weeks. FIG. 5E is a graph showing the amounts of different types of blood cells in a sample obtained from C57BL/6J male mice injected with AAV-GFP or AAV8-FJ1 and then fed a chow diet for 7 weeks. LYM, lymphocytes; MONO, monocytes; GRAN, granulocytes. FIG. 5F are images of heart tissue from C57BL/6J male mice injected with AAV8 containing the cDNA sequence for GFP or F11, then fed a HFD+1-NAME diet for 7 weeks subjected to multiplex-immunohistochemistry. FIG. 5G is a graph quantifying the inflammatory cell infiltration observed in the images in FIG. 5F. FIG. 5H is an image of Masson's trichrome staining of heart tissue, and FIG. 5I is a graph quantifying the fibrosis observed from the same groups of mice. Each point represents a mouse. All data are presented as the mean±SEM. ns, not significant. *P<0.05, **P<0.01, ***P<0.001, and ****p<0.0001 by Student's t test (FIGS. 5A-5C) or by 2-way ANOVA (FIG. 5D).

FIGS. 6A-60 show that FXI activates the BMP-Smad1/5 pathway in the heart. FIG. 3A shows an image of a Western blot and a graph quantifying protein levels in heart tissue from C57BL/6J male mice injected with AAV8-GFP or AAV8-F11 and then fed an HFD+1-NAME diet for 7 weeks. Actin serves as loading control; N=4-8. All data are presented as the mean±SEM. *P<0.05, **P<0.01, ***P<0.001, and ****p<0.0001, by Student's t test (A). For FIGS. 6B-6H, analyzed tissued were obtained from C57BL/6J male mice that received AAV8-GFP or AAV8-F11 and were fed with HFD+1-NAME diet for 7 weeks. Actin served as loading control. FIG. 6B shows the results of a qRT-PCR analysis of BMP7 expression in heart tissue from C57BL/6J male mice fed a chow diet or HFD+1-NAME diet for 7 weeks; N=8. FIG. 6C is a Western blot analysis of p-Smad1/5 levels in white adipose. FIG. 6D is a Western blot analysis of p-Smad1/5 levels in kidney. FIG. 6E is a Western blot analysis of p-Smad1/5 levels in liver. FIG. 6F is a Western blot analysis of p-Smad1/5 levels in brown adipose. FIG. 6G is a Western blot analysis of p-Smad1/5 levels in skeletal muscle. FIG. 6H is a Western blot showing protein levels in heart tissue. Actin served as loading control. N=6. FIG. 6I is a Western blot showing the nuclear fraction of p-Smad1/5 in heart tissue from C57BL/6J male mice injected with AAV8-GFP or AAV8-FJ1 and fed with 7 weeks of HFD+1-NAME diet. Lamin A serves as loading control. N=6. FIG. 6J is a Western blot showing p-Smad1/5 level in heart tissue collected after euthanasia from C57BL/6J male mice injected with control or mouse FXI protein for 2 hours. FIG. 6K is a Western blot showing p-Smad1/5 level in white adipose tissue collected from the same mice in FIG. 6J. FIG. 6L is a Western blot showing p-Smad1/5 level in skeletal muscle tissue collected from the same mice in FIG. 6J. FIG. 6M is a Western blot showing p-Smad1/5 level in lung tissue collected from the same mice in FIG. 6J. For FIGS. 6J-6M, Actin served as loading control. N=4-6. FIG. 6N shows a qRT-PCR analysis Col5a3 mRNA levels in the indicated tissue from C57BL/6J male mice injected with AAV8-GFP or AAV8-F11 and fed an HFD+1-NAME diet for 7 weeks. N=8. FIG. 6O is a qRT-PCR showing mRNA levels of Col5a1 (left) and Adam19 (right) in indicated tissue from C57BL/6J male mice injected with AAV8-GFP or AAV8-F11 and fed with 7 weeks of HFD+1-NAME diet. N=8. All data are presented as the mean±SEM. *P<0.05, **P<0.01, ***P<0.001, and ****p<0.0001, by Student's t test (A). Each point represents a mouse. All data are presented as the mean±SEM. ns, not significant. *P<0.05, **P<0.01, and ***P<0.001, by Student's t test.

FIGS. 7A-7I show that FXI protein activates the BMP-Smad1/5 pathway in cardiomyocytes. FIG. 7A is a Western blot showing p-Smad1/5 expression in neonatal rat ventricular myocytes (NRVMs) treated with control (50% glycerol, 50% water, same as the protein solute) or human FXIa protein (1 μg/mL) with medium containing control or 100 μM phenylephrine (PE) for 24 hours. FIG. 7B quantifies expression of Nppa, Nppb, Adam19, and Col5a3 genes in the NRVMs described in FIG. 7A. For FIGS. 7A and 7B, Actin serves as loading control and N=6. FIG. 7C shows the results of a qRT-PCR analysis of the indicated genes in heart tissue from C57BL/6J male mice fed the chow diet or HFD+1-NAME diet for 7 weeks. N=8. FIG. 7D is a Western blot showing P-Smad1/5 expression in human ES-induced cardiomyocytes treated with control, PE (100 μM) or PE+FXIa protein (1p g/mL) for 24 hours. FIG. 7E shows the results of a qRT-PCR analysis quantifying the expression of Nppa, Nppb, Adam19, and Col5a3 genes in the human ES-induced cardiomyocytes described in FIG. 7D. For FIGS. 7D and 7E, N=4. FIG. 7F is a Western blot analysis of p-Smad1/5 expression levels in 3T3-L1 adipocytes treated with control or human FXIa protein (1 μg/mL) for 24 hours. FIG. 7G is a Western blot analysis of p-Smad1/5 expression levels in HEK293 cells treated with control or human FXIa protein (1 μg/mL) for 24 hours. FIG. 7H is a Western blot analysis of p-Smad1/5 expression levels in Huh7 cells treated with CON or human FXIa protein (1 μg/mL) for 24 hours. FIG. 7I is a Western blot analysis of p-Smad1/5 expression levels in human monocyte-derived macrophages (MDMs) treated with control or human FXIa protein (1 μg/mL) for 24 hours. For FIGS. 7F-7I, Actin served as loading control and N=4. Each point represents a mouse. All data are presented as the mean±SEM. ns, not significant. *P<0.05, **P<0.01, and ***P<0.001, by 2-way ANOVA or Student's t test.

FIGS. 8A-8HH show that FXI overexpression activates BMP signaling to protect against diastolic dysfunction. FIG. 8A is a Western blot showing p-Smad1/5 expression in NRVMs incubated with control or human FXIa protein (1 μg/mL) and DMH1 for 24 hours. ACTIN served as loading control. N=6. For FIGS. 8B-8k, C57BL/6J male mice were injected with AAV8-GFP or AAV8-F11 and DMH1 and then fed an HFD+1-NAME diet for 7 weeks. FIG. 8B comprises graphs of body weight, fat mass and lean mass of the mice. FIG. 8C is a Western blot showing heart p-Smad1/5 expression levels in samples from the mice. FIG. 8D shows the heart weight/tibia length ratio for the mice. FIG. 8E shows the E/e′ ratio for the mice. FIG. 8F shows the LVEF for the mice. For FIGS. 8D-8F, N=8. FIG. 8G comprises representative images of echocardiography performed on the mice. FIG. 8H shows the E/A ratio measured for the mice. FIG. 8I shows the white adipose weight measured for the mice. FIG. 8J shows the blood cells number detected for the mice. FIG. 8K shows the plasma total cholesterol determined for the mice. FIG. 8L is a schematic showing human and mouse FXI sequences. A1-A4 domains and the catalytic domain are shown. The numbering above the domains indicates the range of amino acids from the start to the end of the domain. N and C indicate the N-terminus and C-terminus, respectively. FIG. 8M is a schematic showing the FXI dimer with the A4 domains of each subunit forming the dimer interface. CD=catalytic domain. A1-A4=apple domains 1-4. FIG. 8N is an experimental design for a cell coculture experiment in which Huh7 human liver cells and AML12 mouse liver cells are transfected with respective human or mouse plasmids containing control, wild-type FXI sequence, or FXI with point mutations as indicated in FIG. 8L. Then cells were placed in co-cultures with NRVMs and 3T3-L1 adipocytes for 24 hours. FIG. 8O shows F11 expression in Huh7 human liver cells and AML12 mouse liver cells. FIG. 8P is a Western blot showing p-Smad1/5 protein level in NRVMs. FIG. 8Q is a Western blot showing p-Smad1/5 protein level in 3T3-L1 cells. FIG. 8R shows Col5a3 expression in NRVMs (N=6). For FIGS. 8S-8HH, C57BL/6J male mice were injected with AAV8-GFP, AAV8-F11 or AAV8-F11-Mut (mF11-Mut2) and then fed an HFD+1-NAME diet for 7 weeks (N=5). FIG. 8S shows plasma FXI levels in C57BL/6J male mice. FIG. 8T shows a Western blot analysis and Ponceau S staining of FXI in the plasma of the mice. FIG. 8U shows the body weight measured of the mice. FIG. 8V shows the fat mass measured of the mice. FIG. 8W shows the lean mass measured of the mice. FIG. 8X is a Western blot of heart p-Smad1/5 protein from tissues obtained from the mice (N=6). FIG. 8Y shows the HW/TL determined for the mice. FIG. 8Z shows the E/e′ ratio determined for the mice. FIG. 8AA shows the LVEF measured of the mice. FIG. 8BB shows E/A ratio determined for the mice. FIG. 8CC shows the white adipose weight determined for the mice. FIG. 8DD shows the plasma total cholesterol determined for the mice. FIG. 8EE shows the total blood cells number determined for the mice. FIG. 8FF shows the lymphocytes number determined for the mice. FIG. 8GG shows the number of granulocytes determined for the mice. FIG. 8HH shows the number of monocytes determined for the mice. For FIGS. 8Y-8HH, N=20. Each point represents a mouse. All data are presented as the mean±SEM. ns, not significant. *P<0.05, **P<0.01, and ***P<0.001, by 2-way ANOVA (FIGS. 8C-8F), by 1-way ANOVA (FIGS. 8O, 8R, 8S, and 8U-8HH) or by Student's t test (3A-3C).

FIGS. 9A-9J show that FXI knockout mice exhibit increased diastolic dysfunction in the HFpEF mouse model. FIG. 9A shows a qRT-PCR analysis showing FXI mRNA levels in indicated tissues from heterozygous B6.129X1-F11^{tm1Gjb}/J (F11-Het) mice and wild type (WT) littermates at 8 weeks old that were fed an HFD+1-NAME diet for 7 weeks (N=4). FIG. 9B is a Western blot showing p-Smad1/5 levels in heart tissue from WT mice fed a chow diet (Chow) and WT and F11-Het mice fed an HFD+1-NAME diet for 7 weeks (N=5). FIG. 9C are representative images of and echocardiogram from a WT and an F11-Het mouse. FIG. 9D shows the E/A ratio determined for WT and F11-Het mice. FIG. 9E shows the E/e′ ratio determined for WT and F11-Het mice. FIG. 9F shows the LV mass determined for WT and F11-Het mice. FIG. 9G shows the LVEF determined for WT and F11-Het mice. For FIGS. 9D-9G, mice were examined at baseline (BSL) and after 7 weeks of HFD+1-NAME feeding (HFpEF) (N=8 for WT and N=6 for F11-Het). FIG. 9H shows the HW/TL determined for WT and F11-Het mice. FIG. 9I shows the lung weight (wet/dry ratio) determined for WT and F11-Het mice. FIG. 9J shows the running distance determined for WT and F11-Het mice. For FIGS. 9H-9J, mice were examined after 7 weeks of HFD+1-NAME feeding (HFpEF) (N=8 for WT and N=6 for F11-Het). Each point represents a mouse. All data are presented as the mean±SEM. ns, not significant. *P<0.05, **P<0.01, and ***P<0.001, by 2-way ANOVA (FIGS. 9D-9G), 1-way ANOVA (FIG. 9B), or by Student's t test (FIG. 9A, 9H-9J).

FIG. 10 is an illustration summarizing FXI mediated liver-heart crosstalk in protecting against heart failure.

FIGS. 11A-11CC are full scans of cropped representative blots shown in other figures. FIG. 11A is a full scan of the cropped representative blot shown in FIG. 3D. FIG. 11B is a full scan of the cropped representative blot shown in FIGS. 3G and 3H. FIG. 11C is a full scan of the cropped representative blot shown in FIG. 6H. FIG. 11D is a full scan of the cropped representative blot shown in FIG. 6J. FIG. 11E is a full scan of the cropped representative blot shown in FIG. 6A. FIG. 11F is a full scan of the cropped representative blot shown in FIG. 6I. FIG. 11G is a full scan of the cropped representative blot shown in FIG. 7A. FIG. 11H is a full scan of the cropped representative blot shown in FIG. 7D. FIG. 11I is a full scan of the cropped representative blot shown in FIG. 8A. FIG. 11J is a full scan of the cropped representative blot shown in FIG. 8C. FIG. 11K is a full scan of the cropped representative blot shown in FIG. 8P. FIG. 11L is a full scan of the cropped representative blot shown in FIG. 8X. FIG. 11M is a full scan of the cropped representative blot shown in FIG. 9B. FIG. 11N is a full scan of the cropped representative blot shown in FIG. 1B. FIG. 11O is a full scan of the cropped representative blot shown in FIG. 2C. FIG. 11P is a full scan of the cropped representative blot shown in FIG. 6C. FIG. 11Q is a full scan of the cropped representative blot shown in FIG. 6D. FIG. 11R is a full scan of the cropped representative blot shown in FIG. 6E. FIG. 11S is a full scan of the cropped representative blot shown in FIG. 6F. FIG. 11T is a full scan of the cropped representative blot shown in FIG. 6G. FIG. 11U is a full scan of the cropped representative blot shown in FIG. 6K. FIG. 11V is a full scan of the cropped representative blot shown in FIG. 6L. FIG. 11W is a full scan of the cropped representative blot shown in FIG. 6M. FIG. 11X is a full scan of the cropped representative blot shown in FIG. 7F. FIG. 11Y is a full scan of the cropped representative blot shown in FIG. 7G. FIG. 11Z is a full scan of the cropped representative blot shown in FIG. 7H. FIG. 11AA is a full scan of the cropped representative blot shown in FIG. 7I. FIG. 11BB is a full scan of the cropped representative blot shown in FIG. 8Q. FIG. 11CC is a full scan of the cropped representative blot shown in FIG. 8T.

### DETAILED DESCRIPTION OF THE INVENTION

The present disclosure relates to methods and compositions for the treatment of heart failure with preserved ejection fraction (HFpEF) and is based, at least in part, on the discovery that overexpression of FXI in the liver of a mouse model of HFpEF, attenuates fibrosis, inflammation, and diastolic dysfunction by activating the BMP-Smad1/5 pathway in the heart. In addition, FXI knockout mice exhibited increased diastolic dysfunction in the HFpEF model, which was improved upon FXI overexpression. These observations reveal a novel role for FXI expressed in the liver in protecting the heart from injury, a role distinct from its role in coagulation.

### Definitions

Unless otherwise defined herein, scientific and technical terms used in this application shall have the meanings that are commonly understood by those of ordinary skill in the art. Generally, nomenclature used in connection with, and techniques of, chemistry, cell and tissue culture, molecular biology, cell and cancer biology, neurobiology, neurochemistry, virology, immunology, microbiology, pharmacology, genetics and protein and nucleic acid chemistry, described herein, are those well-known and commonly used in the art.

The methods and techniques of the present disclosure are generally performed, unless otherwise indicated, according to conventional methods well known in the art and as described in various general and more specific references that are cited and discussed throughout this specification. See, e.g., “Principles of Neural Science”, McGraw-Hill Medical, New York, N.Y. (2000); Motulsky, “Intuitive Biostatistics”, Oxford University Press, Inc. (1995); Lodish et al., “Molecular Cell Biology, 4th ed.”, W. H. Freeman & Co., New York (2000); Griffiths et al., “Introduction to Genetic Analysis, 7th ed.”, W. H. Freeman & Co., N.Y. (1999); and Gilbert et al., “Developmental Biology, 6th ed.”, Sinauer Associates, Inc., Sunderland, MA (2000).

Chemistry terms used herein, unless otherwise defined herein, are used according to conventional usage in the art, as exemplified by “The McGraw-Hill Dictionary of Chemical Terms,” Parker S., Ed., McGraw-Hill, San Francisco, C.A. (1985).

All of the above, and any other publications, patents, and published patent applications referred to in this application are specifically incorporated by reference herein. In case of conflict, the present specification, including its specific definitions, will control.

The term “agent” is used herein to denote a chemical compound (such as an organic or inorganic compound, a mixture of chemical compounds), a biological macromolecule (such as a nucleic acid, an antibody, including parts thereof as well as humanized, chimeric and human antibodies and monoclonal antibodies, a protein or portion thereof, e.g., a peptide, a lipid, a carbohydrate), or an extract made from biological materials such as bacteria, plants, fungi, or animal (particularly mammalian) cells or tissues. Agents include, for example, agents whose structure is known, and those whose structure is not known.

“Administering” or “administration of” a substance, a compound or an agent to a subject can be carried out using one of a variety of methods known to those skilled in the art. For example, a compound or an agent can be administered intravenously, arterially, intradermally, intramuscularly, intraperitoneally, subcutaneously, pulmonarily, ocularly, sublingually, orally (by ingestion), intranasally (by inhalation), intraspinally, rectally, intracerebrally, and transdermally (by absorption, e.g., through a skin duct). A compound or agent can also appropriately be introduced by rechargeable or biodegradable polymeric devices or other devices, e.g., patches and pumps, or formulations, which provide for the extended, slow or controlled release of the compound or agent. Administering can also be performed, for example, once, a plurality of times, and/or over one or more extended periods.

Appropriate methods of administering a substance, a compound, or an agent to a subject will also depend, for example, on the age and/or the physical condition of the subject and the chemical and biological properties of the compound or agent (e.g., solubility, digestibility, bioavailability, stability and toxicity). In some embodiments, a compound or an agent is administered parentally, e.g., by injection.

As used herein, the phrase “conjoint administration” refers to any form of administration of two or more different therapeutic agents such that the second agent is administered while the previously administered therapeutic agent is still effective in the body (e.g., the two agents are simultaneously effective in the patient, which may include synergistic effects of the two agents). For example, the different therapeutic compounds can be administered either in the same formulation or in separate formulations, either concomitantly or sequentially. Thus, an individual who receives such treatment can benefit from a combined effect of different therapeutic agents.

A “therapeutically effective amount” or a “therapeutically effective dose” of a drug or agent is an amount of a drug or an agent that when administered to a subject will have the intended therapeutic effect. The full therapeutic effect does not necessarily occur by administration of one dose and may occur only after administration of a series of doses. Thus, a therapeutically effective amount may be administered in one or more administrations. The precise effective amount needed for a subject will depend upon, for example, the subject's size, health and age, and the nature and extent of the condition being treated, such as HFpEF. The skilled worker can readily determine the effective amount for a given situation by routine experimentation.

By “fragment” is meant a portion of a polypeptide or nucleic acid molecule. This portion contains, preferably, at least 10%, 20%, 30%, 40%, 50%, 60%, 70%, 80%, or 90% of the entire length of the reference nucleic acid molecule or polypeptide. A fragment may contain 10, 20, 30, 40, 50, 60, 70, 80, 90, or 100, 200, 300, 400, 500, 600, 700, 800, 900, or 1000 nucleotides or amino acids. Preferred fragments retain some or all of the relevant biological function of the full-length polypeptide, or the polypeptide encoded by the full-length nucleic acid.

The term “modulate” as used herein includes the inhibition or suppression of a function or activity (such as cell proliferation) as well as the enhancement of a function or activity.

The phrase “pharmaceutically acceptable” is art-recognized. In certain embodiments, the term includes compositions, excipients, adjuvants, polymers, and other materials and/or dosage forms that are, within the scope of sound medical judgment, suitable for use in contact with the tissues of human beings and animals without excessive toxicity, irritation, allergic response, or other problem or complication, commensurate with a reasonable benefit/risk ratio.

“Patient,” “subject,” and “individual” are used interchangeably and refer to either a human or a non-human animal. These terms include mammals, such as humans, primates, livestock animals (e.g., bovines, porcines, etc.), companion animals (e.g., canines, felines, etc.) and rodents (e.g., mice and rats). In some embodiments, the subject is a human who experiences one or more symptoms associated with HFpEF.

By “reduces” is meant a negative alteration of at least 10%, 25%, 50%, 75%, or 100%.

A “reference sequence” is a defined sequence used as a basis for sequence comparison. A reference sequence may be a subset of or the entirety of a specified sequence; for example, a segment of a full-length cDNA or gene sequence, or the complete cDNA or gene sequence. For polypeptides, the length of the reference polypeptide sequence will generally be at least about 16 amino acids, preferably at least about 20 amino acids, more preferably at least about 25 amino acids, and even more preferably about 35 amino acids, about 50 amino acids, or about 100 amino acids. For nucleic acids, the length of the reference nucleic acid sequence will generally be at least about 50 nucleotides, preferably at least about 60 nucleotides, more preferably at least about 75 nucleotides, and even more preferably about 100 nucleotides or about 300 nucleotides or any integer thereabout or therebetween.

Nucleic acid molecules useful in the methods of the invention include any nucleic acid molecule that encodes a designated polypeptide or a fragment thereof. Such nucleic acid molecules need not be 100% identical with an endogenous nucleic acid sequence, but will typically exhibit substantial identity. Polynucleotides having “substantial identity” to an endogenous sequence are typically capable of hybridizing with at least one strand of a double-stranded nucleic acid molecule. Nucleic acid molecules useful in the methods of the invention include any nucleic acid molecule that encodes an FXI polypeptide (or other indicated polypeptide) or a fragment thereof. Such nucleic acid molecules need not be 100% identical with an endogenous nucleic acid sequence, but will typically exhibit substantial identity. Polynucleotides having “substantial identity” to an endogenous sequence are typically capable of hybridizing with at least one strand of a double-stranded nucleic acid molecule. By “hybridize” is meant pair to form a double-stranded molecule between complementary polynucleotide sequences (e.g., a gene described herein), or portions thereof, under various conditions of stringency. (See, e.g., Wahl, G. M. and S. L. Berger (1987) Methods Enzymol. 152:399; Kimmel, A. R. (1987) Methods Enzymol. 152:507).

For example, stringent salt concentration will ordinarily be less than about 750 mM NaCl and 75 mM trisodium citrate, preferably less than about 500 mM NaCl and 50 mM trisodium citrate, and more preferably less than about 250 mM NaCl and 25 mM trisodium citrate. Low stringency hybridization can be obtained in the absence of organic solvent, e.g., formamide, while high stringency hybridization can be obtained in the presence of at least about 35% formamide, and more preferably at least about 50% formamide. Stringent temperature conditions will ordinarily include temperatures of at least about 30° C., more preferably of at least about 37° C., and most preferably of at least about 42° C. Varying additional parameters, such as hybridization time, the concentration of detergent, e.g., sodium dodecyl sulfate (SDS), and the inclusion or exclusion of carrier DNA, are well known to those skilled in the art. Various levels of stringency are accomplished by combining these various conditions as needed. In certain preferred embodiments, hybridization will occur at 30° C. in 750 mM NaCl, 75 mM trisodium citrate, and 1% SDS. In more preferred embodiments, hybridization will occur at 37° C. in 500 mM NaCl, 50 nM trisodium citrate, 1% SDS, 35% formamide, and 100 μg/ml denatured salmon sperm DNA (ssDNA). In particularly preferred embodiments, hybridization will occur at 42° C. in 250 mM NaCl, 25 mM trisodium citrate, 1% SDS, 50% formamide, and 200 μg/ml ssDNA. Useful variations on these conditions will be readily apparent to those skilled in the art.

For most applications, washing steps that follow hybridization will also vary in stringency. Wash stringency conditions can be defined by salt concentration and by temperature. As above, wash stringency can be increased by decreasing salt concentration or by increasing temperature. For example, stringent salt concentration for the wash steps will preferably be less than about 30 mM NaCl and 3 mM trisodium citrate, and most preferably less than about 15 mM NaCl and 1.5 mM trisodium citrate. Stringent temperature conditions for the wash steps will ordinarily include a temperature of at least about 25° C., more preferably of at least about 42° C., and even more preferably of at least about 68° C. In certain preferred embodiments, wash steps will occur at 25° C. in 30 mM NaCl, 3 mM trisodium citrate, and 0.1% SDS. In more preferred embodiments, wash steps will occur at 42° C. in 15 mM NaCl, 1.5 mM trisodium citrate, and 0.1% SDS. In particularly preferred embodiments, wash steps will occur at 68° C. in 15 mM NaCl, 1.5 mM trisodium citrate, and 0.1% SDS. Additional variations on these conditions will be readily apparent to those skilled in the art. Hybridization techniques are well known to those skilled in the art and are described, for example, in Benton and Davis (Science 196:180, 1977); Grunstein and Hogness (Proc. Natl. Acad. Sci., USA 72:3961, 1975); Ausubel et al. (Current Protocols in Molecular Biology, Wiley Interscience, New York, 2001); Berger and Kimmel (Guide to Molecular Cloning Techniques, 1987, Academic Press, New York); and Sambrook et al., Molecular Cloning: A Laboratory Manual, Cold Spring Harbor Laboratory Press, New York.

By “substantially identical” is meant a polypeptide or nucleic acid molecule exhibiting at least 50% identity to a reference amino acid sequence (for example, any one of the amino acid sequences described herein) or nucleic acid sequence (for example, any one of the nucleic acid sequences described herein). Preferably, such a sequence is at least 60%, more preferably at least 80% or 85%, and more preferably at least 90%, 95% or even at least 99% identical at the amino acid level or nucleic acid to the sequence used for comparison.

Sequence identity is typically measured using sequence analysis software (for example, Sequence Analysis Software Package of the Genetics Computer Group, University of Wisconsin Biotechnology Center, 1710 University Avenue, Madison, Wis. 53705, BLAST, BESTFIT, GAP, or PILEUP/PRETTYBOX programs). Such software matches identical or similar sequences by assigning degrees of homology to various substitutions, deletions, and/or other modifications. Conservative substitutions typically include substitutions within the following groups: glycine, alanine; valine, isoleucine, leucine; aspartic acid, glutamic acid, asparagine, glutamine; serine, threonine; lysine, arginine; and phenylalanine, tyrosine. In an exemplary approach to determining the degree of identity, a BLAST program may be used, with a probability score between e-3 and e-100 indicating a closely related sequence.

Ranges provided herein are understood to be shorthand for all of the values within the range. For example, a range of 1 to 50 is understood to include any number, combination of numbers, or sub-range from 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50.

“Treating” a condition or patient refers to taking steps to obtain beneficial or desired results, including clinical results. As used herein, and as well understood in the art, “treatment” is an approach for obtaining beneficial or desired results, including clinical results. Beneficial or desired clinical results can include, but are not limited to, alleviation or amelioration of one or more symptoms or conditions, diminishment of extent of disease, stabilized (i.e., not worsening) state of disease, preventing spread of disease, delay or slowing of disease progression, amelioration or palliation of the disease state, and remission (whether partial or total), whether detectable or undetectable. “Treatment” can also mean prolonging survival as compared to expected survival if not receiving treatment.

As used herein, the terms “treat,” treating,” “treatment,” and the like refer to reducing or ameliorating a disorder and/or symptoms associated therewith. It will be appreciated that, although not precluded, treating a disorder or condition does not require that the disorder, condition, or symptoms associated therewith be completely eliminated.

As used herein, a therapeutic that “prevents” a disorder or condition refers to a compound that, in a statistical sample, reduces the occurrence of the disorder or condition in the treated sample relative to an untreated control sample, or delays the onset or reduces the severity of one or more symptoms of the disorder or condition relative to the untreated control sample.

Unless specifically stated or obvious from context, as used herein, the term “or” is understood to be inclusive. Unless specifically stated or obvious from context, as used herein, the terms “a,” “an,” and “the” are understood to be singular or plural.

Unless specifically stated or obvious from context, as used herein, the term “about” is understood as within a range of normal tolerance in the art, for example within 2 standard deviations of the mean. About can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein are modified by the term about.

### Recombinant Polypeptide Expression

Recombinant FXI polypeptides or fragments thereof are contemplated herein. Suh recombinant proteins can be expressed from an engineered nucleic acid. A nucleic acid encoding an FXI polypeptide or fragment thereof can be inserted into an appropriate expression vector by techniques well known in the art. For example, a double stranded DNA can be cloned into a suitable vector by restriction enzyme linking involving the use of synthetic DNA linkers or by blunt-ended ligation. DNA ligases are usually used to ligate the DNA molecules and undesirable joining can be avoided by treatment with alkaline phosphatase.

The invention includes vectors (e.g., recombinant plasmids) that include nucleic acid molecules (e.g., genes or recombinant nucleic acid molecules encoding genes) as described herein. The term “recombinant vector” includes a vector (e.g., plasmid, phage, phasmid, virus, cosmid, fosmid, or other purified nucleic acid vector) that has been altered, modified or engineered such that it contains greater, fewer or different nucleic acid sequences than those included in the native or natural nucleic acid molecule from which the recombinant vector was derived. A recombinant vector may include a nucleotide sequence encoding an FXI polypeptide or fragment thereof operatively linked to a regulatory sequence, e.g., a promoter sequence, terminator sequence, and the like. Recombinant vectors that allow for expression of the genes or nucleic acids included in them are referred to as “expression vectors.”

In some of the molecules of the invention described herein, one or more DNA molecules having a nucleotide sequence encoding one or more polypeptides of the invention are operatively linked to one or more regulatory sequences, which are capable of integrating the desired DNA molecule into a prokaryotic host cell. Cells which have been stably transformed by the introduced DNA can be selected, for example, by introducing one or more markers which allow for selection of host cells which contain the expression vector. A selectable marker gene can either be linked directly to a nucleic acid sequence to be expressed, or be introduced into the same cell by co-transfection. Additional elements may also be needed for optimal synthesis of proteins described herein. It would be apparent to one of ordinary skill in the art which additional elements to use.

Factors of importance in selecting a particular plasmid or viral vector include, but are not limited to, the ease with which recipient cells that contain the vector are recognized and selected from those recipient cells that do not contain the vector; the number of copies of the vector that are desired in a particular host; and whether it is desirable to be able to “shuttle” the vector between host cells of different species.

Once the vector(s) is constructed to include a DNA sequence for expression, it may be introduced into an appropriate host cell by one or more of a variety of suitable methods that are known in the art, including but not limited to, transformation, transfection, conjugation, protoplast fusion, electroporation, calcium phosphate-precipitation, direct microinjection, etc.

After the introduction of one or more vector(s), host cells are usually grown in a selective medium that selects for the growth of vector-containing cells. Expression of recombinant proteins can be detected by immunoassays including Western blot analysis and immunofluorescence. Purification of recombinant proteins can be carried out by any of the methods known in the art or described herein, for example, any conventional procedures involving extraction, precipitation, chromatography and electrophoresis. A further purification procedure that may be used for purifying proteins is affinity chromatography using monoclonal antibodies that bind a target protein. Generally, crude preparations containing a recombinant protein are passed through a column on which a suitable monoclonal antibody is immobilized. The protein binds to the column via the specific antibody while the impurities pass through. After washing the column, the protein is eluted by changing pH or ionic strength.

### Polynucleotide Delivery

Polynucleotides encoding an FXI polypeptide or a fragment thereof can be delivered to a subject in need thereof to induce, promote, enhance, or otherwise modulate expression of the FXI polypeptide or fragment thereof. In some embodiments, the delivery of the polynucleotide encoding an FXI polypeptide or fragment thereof, results in a therapeutic benefit to the subject. For example, a polynucleotide encoding an FXI polypeptide or a fragment thereof can be administered to a subject to treat heart failure (e.g., HFpEF).

Methods of delivering nucleic acids to a subject or a cell are known in the art. In one aspect, a method is provided for delivering a nucleic acid molecule encoding an FXI protein or fragment thereof to a subject. The nucleic acid encoding the FXI polypeptide or fragment thereof can be incorporated into a viral vector. Viruses, also referred to as viral particles, comprising viral vectors that have been modified to comprise the nucleic acid sequence of interest can be administered to a subject in need thereof. In some embodiments about 10⁷, 10⁸, 10⁹, 10¹⁰, 10¹¹, 10¹², 10¹³, 10¹⁴ or more viral particles viral particles can be administered to a subject. In some embodiments, between about 10⁷ and 10¹⁴, between about 10⁷ and 10¹³, between about 10⁷ and 10¹², between about 10⁷ and 10¹¹, between about 10⁷ and 10¹⁰, between about 10⁷ and 10⁹, between about 10⁸ and 10¹⁴, between about 10⁹ and 10¹⁴, between about 10¹⁰ and 10¹⁴, between about 10¹¹ and 10¹⁴, or between about 10¹² and 10¹⁴ viral particles are administered to the subject. The viral particles can be suspended within a suitable volume (e.g., 10 μL, 50 μL, 100 μL, 500 μL, or 1000 μL) for administration.

As described herein, an adeno-associated virus (AAV) can efficiently deliver nucleic acids (e.g., polynucleotides encoding an FXI polypeptide or fragment thereof) to a cell. As demonstrated herein, expression of an FXI polynucleotide delivered using an AAV-vector can result in improved heart function (e.g., diastolic function) in a subject. In some embodiments, the AAV vector is an AAV8 vector.

The viral vector can comprise regulatory sequences that restrict expression or preferentially express a transgene (e.g., F11) or fragment thereof from the vector in certain cells. For example, the viral vector can comprise regulatory sequences that preferentially express the transgene in liver cells.

Expression of FXI from a vector or other polynucleotides described herein may be directed by a heterologous promoter. As used herein, a “heterologous promoter” refers to a promoter that does not naturally direct expression of the coding sequence in the plasmid, vector, etc. (i.e., is not found with the particular coding sequence in nature).

Non-viral approaches can also be employed to introduce a polynucleotide encoding an FXI polypeptide or fragment thereof to a cell of a subject in need thereof. For example, a nucleic acid molecule can be introduced into a cell by administering the nucleic acid via lipofection.

Polynucleotides encoding an FXI polypeptide or fragment thereof can be introduced into a cell in vitro. For example, a polynucleotide can be introduced into a cell via transfection. Such methods can use calcium phosphate, DEAE dextran, electroporation, and protoplast fusion to facilitate the transfection. Liposomes can also be potentially beneficial for delivery of DNA into a cell. Transplantation of normal genes into the affected tissues of a patient can also be accomplished by transferring a normal nucleic acid into a cultivatable cell type ex vivo (e.g., an autologous or heterologous primary cell or progeny thereof), after which the cell (or its descendants) are injected into a targeted tissue.

### Methods of Treatment

The methods and compositions disclosed herein relate to the treatment, prevention, and/or modulation of heart failure. Heart failure occurs when the heart muscle is incapable of pumping sufficient blood to the body. Heart failure is typically a chronic and progressive disease, most often observed in older individuals or individuals having underlying conditions (e.g., obesity, smoking-related issues, diabetes, kidney disease, etc.). Approximately 50% of all heart failure patients have preserved ejection fraction.

One aspect of the present disclosure provides a method of treating HFpEF by administering an FXI polypeptide or fragment thereof or a nucleic acid encoding an FXI polypeptide or fragment thereof a subject. In certain aspects, the present disclosure provides methods of ameliorating one or more symptoms of heart failure in a subject by administering an FXI polypeptide or fragment thereof or a nucleic acid encoding an FXI polypeptide or fragment thereof to a subject having or suspected of having heart failure. Symptoms can vary from subject to subject; thus, ascertaining the severity of a subject's HfpEF at different times during treatment can assess the effect of administering the FXI polypeptide or fragment thereof or nucleic acid encoding an FXI polypeptide or fragment thereof on the subject's heart failure.

In the methods disclosed herein, the FXI polypeptide or fragment thereof or nucleic acid encoding an FXI polypeptide or fragment thereof can be conjointly administered with an additional agent. The additional agent and the FXI polypeptide or fragment thereof or nucleic acid encoding an FXI polypeptide or fragment thereof can be used to treat a subject's heart failure and/or ameliorate at least one symptom of the subject's heart failure. In some embodiments, the efficacy of the conjoint therapy can be assessed in the same manner as administering only the FXI polypeptide or fragment thereof or nucleic acid encoding an FXI polypeptide or fragment thereof as described above (i.e., ascertaining the severity of a subject's HfpEF at different times during treatment, e.g., prior to and post administration of the one or more of the agents in the combination therapy). In some embodiments, the FXI polypeptide or fragment thereof or nucleic acid encoding an FXI polypeptide or fragment thereof and the additional agent are administered simultaneously or sequentially.

In some embodiments, the additional agent is a hepatocyte growth factor activator (HGFAC) when overexpressed increased LV mass and complement C8 gamma chain (C8G) polypeptide or fragment thereof or a nucleotide encoding an HGFAC or C8G polypeptide or fragment thereof. In some embodiments, the additional agent is phenylephrine (PE) or dorsomorphin homolog 1 (DMH1).

### Screening Methods

One aspect of the present invention relates to screening assays that identify if a subject's heart failure is likely to respond to FXI administration. Screening assays may also be used to identify agents, in combination with FXI, that treat, prevent, or otherwise modulate (e.g., reduce symptoms) celiac disease. Identifying such an agent involves determining the ability of the agent to treat, prevent, or otherwise modulate heart failure (e.g., HFpEF), for example, by monitoring the severity, progression, development, reduction, or elimination of a subject's symptoms. In some embodiments, ejection fraction is measured in a subject. In some embodiments, the level of FXI expression (e.g., mRNA, protein or both) is measured.

In another aspect, the effectiveness of treating a subject's heart failure (HFpEF) by administering an FXI polypeptide or fragment thereof or a nucleic acid molecule encoding an FXI polypeptide is assessed. Assessing the effectiveness of the treatment can be incorporate a method known in the art or by comparing FXI expression levels before or after administration of the FXI polypeptide or fragment thereof or a nucleic acid molecule encoding an FXI polypeptide. For example, the presence and/or severity of a subject's heart disease can be determined at a first time point (e.g., prior to administration of the FXI polypeptide or fragment thereof or the nucleic acid molecule encoding an FXI polypeptide) and at a second time point (e.g., post-administration). Detecting the presence and/or determining the severity of a subject's heart failure can be accomplished by using any number of techniques to assess standard criteria. Such techniques include, but are not limited to, enteroscopic examination, small bowel imaging, immunohistochemistry, flow cytometry, blood and tissue sample analysis, and molecular genetics. Additionally, immunoassays, PCR (e.g., RT-PCR and qPCR), chromosomal analysis, biomarker analysis, and physical examination of a subject can be used in assessing a subject.

### Methods of Administration

An FXI polypeptide or fragment thereof or a nucleic acid molecule encoding an FXI polypeptide or fragment thereof can be administered in any form to a subject having heart failure, although it is often formulated for intravenous, subcutaneous, and/or intraperitoneal administration. Other means of administration are contemplated herein. For example, administration may be accomplished by parenteral, intravenous, intra-arterial, intramuscular, intraventricular, rectal, pulmonary, or intranasal administration. In some embodiments, between about 1 mg and about 50 mg; between about 1 mg and about 25 mg, between about 1 mg and about 10 mg, and between about 1 mg and 5 mg of FXI polypeptide is administered to a subject suspected of having HFpEF disease. In some embodiments, between about 5 mg and about 50 mg, between about 10 mg and about 50 mg, or between about 25 and about 50 mg of an FXI polypeptide is administered to a subject having or suspected of having HFpEF.

An FXI polypeptide or fragment thereof or a nucleic acid encoding an FXI polypeptide or fragment thereof can be administered one or more times a day. For example, a subject may be administered an FXI polypeptide or fragment thereof or a nucleic acid encoding an FXI polypeptide or fragment thereof one, two, three, or even four times a day. In certain preferred embodiments, the FXI polypeptide or nucleic acid encoding an FXI polypeptide is administered twice daily in 10-mg doses or 5-mg doses, e.g., depending on the severity of the condition and the patient's response to the initial dosage. In some embodiments, the FXI polypeptide or nucleic acid encoding an FXI polypeptide is administered in multiple equal doses.

The present invention also pertains to monitoring the influence of administration of an FXI polypeptide or fragment thereof or a nucleic acid encoding an FXI polypeptide or fragment thereof, alone or in combination with one or more additional therapeutic agents, on HFpEF. For example, monitoring the influence of FXI administration on a subject's HFpEF can comprise performing echocardiograms during a course of treatment to determine changes in heart function in response to treatment. In some embodiments, a first echocardiogram is acquired at or near the time treatment is commenced, and heart function observed from this first test serves as a reference to which later-acquired echocardiograms can be compared.

### Pharmaceutical Compositions

The compositions and methods of the present invention may be utilized to treat an individual in need thereof. In certain embodiments, the individual is a mammal such as a human, or a non-human mammal. When administered to an animal, such as a human, the composition or the compound is preferably administered as a pharmaceutical composition comprising, for example, a compound of the invention and a pharmaceutically acceptable carrier. Pharmaceutically acceptable carriers are well known in the art and include, for example, aqueous solutions such as water or physiologically buffered saline or other solvents or vehicles such as glycols, glycerol, oils such as olive oil, or injectable organic esters. In preferred embodiments, when such pharmaceutical compositions are for human administration, particularly for invasive routes of administration (i.e., routes, such as injection or implantation, that circumvent transport or diffusion through an epithelial barrier), the aqueous solution is pyrogen-free, or substantially pyrogen-free. The excipients can be chosen, for example, to effect delayed release of an agent or to selectively target one or more cells, tissues, or organs. The pharmaceutical composition can be in dosage unit form such as tablet, capsule (including sprinkle capsule and gelatin capsule), granule, lyophilisate for reconstitution, powder, solution, syrup, suppository, injection or the like. The composition can also be present in a transdermal delivery system, e.g., a skin patch. The composition can also be present in a solution suitable for topical administration, such as a lotion, cream, or ointment.

The phrase “pharmaceutically acceptable carrier” as used herein means a pharmaceutically acceptable material, composition or vehicle, such as a liquid or solid filler, diluent, excipient, solvent or encapsulating material. Each carrier must be “acceptable” in the sense of being compatible with the other ingredients of the formulation and not injurious to the patient. Some examples of materials which can serve as pharmaceutically acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

A pharmaceutically acceptable carrier can contain physiologically acceptable agents that act, for example, to stabilize, increase solubility or to increase the absorption of a compound such as an FXI polypeptide or a nucleic acid encoding an FXI polypeptide. Such physiologically acceptable agents include, for example, carbohydrates, such as glucose, sucrose or dextrans, antioxidants, such as ascorbic acid or glutathione, chelating agents, low molecular weight proteins or other stabilizers or excipients. The choice of a pharmaceutically acceptable carrier, including a physiologically acceptable agent, depends, for example, on the route of administration of the composition. The preparation or pharmaceutical composition can be a self-emulsifying drug delivery system or a self-microemulsifying drug delivery system. The pharmaceutical composition (preparation) also can be a liposome or other polymer matrix, which can have incorporated therein, for example, a compound of the invention. Liposomes, for example, which comprise phospholipids or other lipids, are nontoxic, physiologically acceptable and metabolizable carriers that are relatively simple to make and administer.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

A pharmaceutical composition (preparation) can be administered to a subject by any of a number of routes of administration including, for example, orally (for example, drenches as in aqueous or non-aqueous solutions or suspensions, tablets, capsules (including sprinkle capsules and gelatin capsules), boluses, powders, granules, pastes for application to the tongue); absorption through the oral mucosa (e.g., sublingually); subcutaneously; rectally; intranasally; by inhalation; transdermally (for example as a patch applied to the skin); and topically (for example, as a cream, ointment or spray applied to the skin). The compound may also be formulated for inhalation. In certain embodiments, a compound may be simply dissolved or suspended in sterile water. Details of appropriate routes of administration and compositions suitable for same can be found in, for example, U.S. Pat. Nos. 6,110,973; 5,763,493; 5,731,000; 5,541,231; 5,427,798; 5,358,970; and 4,172,896, as well as in patents cited therein.

The formulations may conveniently be presented in unit dosage form and may be prepared by any methods well known in the art of pharmacy. The amount of active ingredient which can be combined with a carrier material to produce a single dosage form will vary depending upon the host being treated, the particular mode of administration. The amount of active ingredient that can be combined with a carrier material to produce a single dosage form will generally be that amount of the compound that produces a therapeutic effect. Generally, this amount will range from about 1 percent to about ninety-nine percent of active ingredient, preferably from about 5 percent to about 70 percent, most preferably from about 10 percent to about 30 percent.

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal and intrasternal injection and infusion. Pharmaceutical compositions suitable for parenteral administration comprise one or more active compounds in combination with one or more pharmaceutically acceptable sterile isotonic aqueous or nonaqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents.

Examples of suitable aqueous and nonaqueous carriers that may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of the action of microorganisms may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents that delay absorption such as aluminum monostearate and gelatin.

The subject receiving this treatment is any animal in need, including primates, in particular humans, and animal models of HFpEF.

In certain embodiments, compounds of the invention may be used alone or conjointly administered with another type of therapeutic agent.

### Kits

The invention provides kits for the treatment or prevention of heart failure (i.e., HfpEF). In some embodiments, the kit includes a therapeutic composition containing an FXI polypeptide or fragment thereof or a polynucleotide encoding an FX polypeptide or fragment thereof. The kit can also comprise containers for the therapeutic composition. Such containers can be boxes, ampoules, bottles, vials, tubes, bags, pouches, blister-packs, or other suitable container forms known in the art. Such containers can be made of plastic, glass, laminated paper, metal foil, or other materials suitable for holding medicaments.

If desired, a pharmaceutical composition of the invention is provided together with instructions for administering the pharmaceutical composition to a subject having or at risk of developing heart failure with preserved ejection fraction (HFpEF). The instructions will generally include information about the use of the composition for the treatment or prevention of HFpEF. In some embodiments, the instructions include at least one of the following: description of the therapeutic agent; dosage schedule and administration for treatment or prevention of cancer or symptoms thereof; precautions; warnings; indications; counter-indications; over dosage information; adverse reactions; animal pharmacology; clinical studies; and/or references. The instructions may be printed directly on the container (when present), or as a label applied to the container, or as a separate sheet, pamphlet, card, or folder supplied in or with the container.

### EXAMPLES

The invention now being generally described, it will be more readily understood by reference to the following examples which are included merely for purposes of illustration of certain aspects and embodiments of the present invention and are not intended to limit the invention.

### Example 1: Materials and Methods

### Animals

All animal experiments were approved by the University of California Los Angeles (UCLA) Animal Care and Use Committee, in accordance with Public Health Service guidelines. Mice were maintained on a 12-h light/dark cycle from 6 am to 6 pm. Wild-type C57BL/6J mice (Stock No: 000664) and B6.129X1-F11^{tm1Gjb}/J (Stock No: 030987) were obtained from the Jackson Laboratory. 100 strains of inbred mice included in the Hybrid Mouse Diversity Panel (HMDP) were obtained from the Jackson Laboratory and have been described in detail²⁸. HFpEF was induced by high fat diet (HFD, Research Diet #D12492) and No-Nitro-L-arginine methyl ester hydrochloride (1-NAME, Sigma #N5751-25G) feeding for 7 weeks¹². 1-NAME was dissolved in drinking water (0.5 g/L, pH=7.4). DMH1 (Cayman Chemical #16679) was dissolved in 44% w/v aqueous (2-hydroxypropyl-β)-cyclodextrin (Sigma-Aldrich, #H5784) and i.p. injected into the mice at 3 mg/kg body weight every other day from the injection of AAV8 until mice sacrifice.

### Cell Culture

Neonatal rat ventricular myocytes (NRVMs) were isolated from P1-P3 day old Sprague-Dawley rat pups as described previously with modifications²⁹. Briefly, rat left ventricles were isolated and digested with collagenase, and the resulting cell slurry was fractionated on a Percoll gradient by centrifugation. The myocyte-rich fraction was isolated, washed and plated in Dulbecco's modified Eagle's medium (DMEM; Gibco) supplemented with 5% horse serum, 15 mM HEPES and 1% penicillin/streptomycin. 24 hours after resting in plating medium, the NRVMs were then changed to serum-free medium for further experiments. 3T3-L1, Huh7 and HEK-293 cells were maintained in DMDM medium supplemented with 10% fetal bovine serum and 1% penicillin/streptomycin. AML12 cells were maintained in DMDM/F12 medium supplemented with 10% fetal bovine serum, 1% penicillin/streptomycin and insulin-transferrin-selenium (100×).

Human embryonic stem cells-derived cardiomyocytes (hES-CMs) were differentiated from hES cell line H9 as previously described³⁰. Briefly, hES cells were maintained in mTeSR1 medium (stemcell technology) and differentiation was induced in RPMI 1640 supplemented with B27 minus insulin (Invitrogen). On day 0-1, the medium was supplemented with 6 μM CHIR99021 (selleckchem). Between day 3-5, cells were incubated with the medium containing 5 μM IWR1 (Sigma-Aldrich). After day 7, the medium was replaced with RPMI 1640 plus B27 maintain medium. From day 10-11, RPMI 1640 without D-glucose supplemented with B27 was transiently used for metabolic purification of CMs. hES-CMs were incubated with FXIa protein and 100 μM phenylephrine (PE) for 24 hrs and cells were harvested for analysis.

Human monocyte-derived macrophages (MDMs) were derived from human peripheral blood mononuclear cells (PBMCs) of healthy donors. Human monocytes were isolated from PBMCs by adherence. Briefly, PBMCs were suspended in serum-free RPMI 1640 media (Corning Cellgro, Cat #10-040-CV) at 10×10⁶ cells/ml. 12.5 ml of cell suspension were added to each 10-cm dish and incubated in 5% CO₂ incubator for one hour. Dishes were washed twice and adherent monocytes were cultured in complete RPMI 1640 media with human M-CSF (Peprotech, 300-25, 10 ng/ml) for 6 days to generate MDMs. At day 6, MDMs were collected and reseeded in a 6-well plate in complete RPMI 1640 media and treated with FXIa protein.

### Coculture Experiments

Cells were cultured under conditions as described above. Experiments were carried out in a transwell system (Corning #07-200-170). On day 1, Huh7 and AML12 hepatocytes were plated into the culture insert, respectively, and allowed to achieve confluency. On day 2, hepatocytes were then transfected with GFP, human F11 (hF11), mouse F11 (mF11) or mutant F11 plasmids using lipofectamine 3000 reagent (Thermo Fisher #L3000008) in accordance with the recommended protocol. NRVMs or 3T3-L1 adipocytes were plated into a separate plate at 80% confluence. On day 3, NRVMs were switched to serum-free medium supplemented with 100 μM phenylephrine (PE). Hepatocytes inserts were added to NRVMs or 3T3-L1 cells for 24 hours, and then cells were harvested for RNA and protein extraction.

### Plasmids

Human F11_pCMV6-Entry-Myc-DDK expression plasmid (Accession No.: NM_000128, ORF sequence 1875 bp) was obtained from OriGene (#RC213056). Two mutations (GTT to ATT and CCC to CTC) were introduced into human F11 plasmid (GenScript). Mouse F11_pcDNA3.1+/C-(K)-DYK (Accession No.: NM_028066.2, ORF sequence 1872 bp) was obtained from GenScript (Clone ID: OMu22400D). Two mutations (GTA to ATA and CCA to CTA) were introduced into mouse F11 plasmid (GenScript). GFP vectors were used as control.

### Adeno-Associated Viruses (AAV)

AAV8-TBG-eGFP (VB1743), and AAV8-TBG-M-F11 (AAV-258829) were obtained from Vector Biolabs. For AAV8-TBG-mF11-Mut2, mF11-Mut2 sequence from mouse F11 Mut2_pcDNA3.1+/C-(K)-DYK plasmid was subcloned into pAAV-TBG vector (Vector Biolabs). Virus was diluted with saline and 100 μL of virus was i.p. injected into each mouse (5×10¹¹ gc/mouse titer).

### Factor XI Proteins

Human Factor XIa protein was obtained from Abcam (#ab62411) and recombinant mouse FXI protein was obtained from OriGene (#TP509529). NRVMs were plated at 80% confluence. The next day, NRVMs were switched to serum-free medium supplemented with 100 μM phenylephrine (PE) and treated with control or human FXIa protein (1 μg/mL). 24 hours after treatment, cells were harvested for protein and total RNA extraction. Human ES-induced cardiomyocytes (hES-CMs) were seeded at 90% confluence one day before treatment. The next day, hES-CMs were changed to fresh medium supplemented with 100 μM phenylephrine (PE) and treated with control or human FXIa protein (1 μg/mL) for 24 hours. For in vivo injection, mouse FXI protein was diluted in sterile saline and administrated through tail vein injection (8 μg/100 μL/mouse). 2 hours after injection, mice were sacrificed and tissues were collected for western blotting.

### Transthoracic Echocardiography

The mice were anesthetized and maintained with 1-2% isoflurane in 95% oxygen. Transthoracic echocardiography was conducted with Vevo 2100 high-frequency, high-resolution digital imaging system (VisualSonics) equipped with a MS400 MicroScan Transducer. A parasternal short axis view was used to obtain M-mode images for analysis of fractional shortening, ejection fraction, and other cardiac functional parameters. Apical four-chamber view was used to obtain tissue Doppler imaging (TDI) mode and Pulse-wave Doppler (PWD) mode for analysis of myocardial velocity and blood flow velocity, respectively. Echocardiographic results in the different groups of mice are listed in Table 1.

TABLE 1
Echocardiography in the different groups of mice

| Condition | MV E/A | MV E/e′ | EF | FS | LV Mass | LV Vol; d | LV Vol; s |
|---|---|---|---|---|---|---|---|
| Chow-F11-baseline | 1.511 ± 0.033 | 20.502 ± 1.288 | 58.467 ± 1.643 | 30.326 ± 1.177 | 93.261 ± 4.183 | 56.606 ± 3.262 | 23.209 ± 0.860 |
| HFpEF-GFP-baseline | 1.437 ± 0.041 | 21.700 ± 0.843 | 59.694 ± 1.461 | 31.264 ± 1.035 | 91.607 ± 3.517 | 62.439 ± 3.548 | 25.119 ± 1.733 |
| HFpEF-F11-baseline | 1.526 ± 0.049 | 21.313 ± 0.676 | 60.035 ± 1.085 | 31.539 ± 0.756 | 94.221 ± 2.052 | 67.294 ± 3.073 | 26.939 ± 1.516 |
| Chow-GFP-4w | 1.534 ± 0.049 | 21.134 ± 1.176 | 60.264 ± 2.163 | 31.657 ± 1.478 | 94.488 ± 5.184 | 59.645 ± 4.644 | 24.030 ± 2.81 |
| Chow-F11-4w | 1.563 ± 0.039 | 20.712 ± 1.111 | 62.861 ± 1.137 | 33.321 ± 0.832 | 90.823 ± 4.634 | 55.865 ± 3.882 | 20.751 ± 1.601 |
| HFpEF-GFP-4w | 2.319 ± 0.120 | 39.962 ± 1.698 | 58.762 ± 1.536 | 30.851 ± 1.085 | 132.130 ± 3.716 | 76.870 ± 4.154 | 31.619 ± 1.998 |
| HFpEF-F11-4w | 1.928 ± 0.120 | 31.520 ± 1.307 | 58.541 ± 1.588 | 30.798 ± 1.089 | 128.270 ± 4.771 | 83.855 ± 3.280 | 34.786 ± 1.994 |
| Chow-GFP-7w | 1.554 ± 0.069 | 22.383 ± 0.965 | 62.371 ± 2.555 | 33.215 ± 1.885 | 94.808 ± 3.142 | 57.777 ± 4.213 | 21.832 ± 2.342 |
| Chow-F11-7w | 1.600 ± 0.046 | 21.143 ± 1.036 | 63.074 ± 1.734 | 33.597 ± 1.308 | 98.389 ± 4.573 | 58.531 ± 4.250 | 21.476 ± 1.696 |
| HFpEF-GFP-7w | 2.314 ± 0.152 | 45.078 ± 1.894 | 64.362 ± 1.635 | 35.205 ± 1.308 | 135.489 ± 5.703 | 76.418 ± 3.585 | 27.44 ± 2.013 |
| HFpEF-F11-7w | 1.887 ± 0.083 | 36.426 ± 1.681 | 63.925 ± 2.696 | 34.875 ± 2.055 | 128.711 ± 5.251 | 79.395 ± 3.437 | 29.022 ± 2.725 |
| HFpEF-Hgfac-7w | 1.585 ± 0.095 | 35.336 ± 2.286 | 63.332 ± 2.149 | 34.412 ± 1.639 | 154.698 ± 6.749 | 81.108 ± 4.974 | 29.924 ± 2.776 |
| HFpEF-C8g-7w | 1.582 ± 0.137 | 33.748 ± 2.321 | 68.141 ± 2.512 | 37.799 ± 1.994 | 133.755 ± 6.773 | 63.815 ± 3.043 | 20.687 ± 2.335 |
| GFP + CON-baseline | 1.450 ± 0.045 | 21.285 ± 0.983 | 59.364 ± 1.730 | 31.062 ± 1.225 | 89.629 ± 4.130 | 63.577 ± 4.284 | 25.798 ± 2.121 |
| F11 + CON-baseline | 1.441 ± 0.053 | 22.497 ± 0.718 | 58.653 ± 0.951 | 30.567 ± 0.670 | 97.645 ± 1.491 | 67.349 ± 3.926 | 27.813 ± 1.676 |
| GFP + DMH1-baseline | 1.500 ± 0.058 | 21.598 ± 0.927 | 61.875 ± 1.572 | 32.811 ± 1.107 | 91.737 ± 3.687 | 63.54 ± 1.712 | 24.227 ± 1.197 |
| F11 + DMH1-baseline | 1.416 ± 0.035 | 22.053 ± 0.592 | 57.527 ± 0.859 | 29.748 ± 0.609 | 93.292 ± 3.688 | 64.33 ± 4.667 | 27.237 ± 1.943 |
| GFP + CON-4w | 2.281 ± 0.115 | 39.977 ± 1.336 | 62.251 ± 2.681 | 33.266 ± 1.872 | 118.540 ± 5.208 | 65.274 ± 3.437 | 24.942 ± 2.571 |
| F11 + CON-4w | 1.881 ± 0.084 | 34.377 ± 1.012 | 60.606 ± 1.379 | 32.063 ± 0.994 | 124.540 ± 6.360 | 74.963 ± 4.988 | 29.708 ± 2.470 |
| GFP + DMH1-4w | 2.172 ± 0.145 | 39.431 ± 1.185 | 57.560 ± 2.324 | 30.063 ± 1.584 | 118.120 ± 4.439 | 73.889 ± 4.234 | 31.472 ± 2.412 |
| F11 + DMH1-4w | 2.140 ± 0.100 | 37.899 ± 1.165 | 60.341 ± 1.747 | 32.044 ± 1.223 | 132.330 ± 7.729 | 85.575 ± 7.631 | 34.232 ± 3.827 |
| GFP + CON-7w | 2.379 ± 0.121 | 43.496 ± 1.352 | 63.514 ± 1.896 | 34.277 ± 1.354 | 133.780 ± 4.031 | 79.462 ± 5.057 | 28.907 ± 2.049 |
| F11 + CON-7w | 1.988 ± 0.081 | 37.516 ± 1.888 | 61.449 ± 2.252 | 32.762 ± 1.627 | 117.820 ± 4.851 | 73.360 ± 3.387 | 28.678 ± 2.641 |
| GFP + DMH1-7w | 2.331 ± 0.123 | 42.464 ± 1.454 | 57.756 ± 1.625 | 30.140 ± 1.065 | 124.630 ± 7.174 | 77.578 ± 5.968 | 33.167 ± 3.560 |
| F11 + DMH1-7w | 2.466 ± 0.086 | 42.810 ± 0.910 | 59.721 ± 1.256 | 31.425 ± 0.882 | 119.970 ± 2.382 | 73.602 ± 2.582 | 29.565 ± 1.087 |
| GFP-7w | 2.420 ± 0.160 | 40.901 ± 1.745 | 55.151 ± 2.162 | 28.513 ± 1.429 | 125.136 ± 4.068 | 77.158 ± 3.528 | 35.076 ± 2.792 |
| F11-7w | 1.854 ± 0.140 | 29.567 ± 1.710 | 58.035 ± 3.336 | 30.563 ± 2.363 | 124.452 ± 9.168 | 83.791 ± 5.617 | 34.834 ± 2.835 |
| F11-Mut-7w | 2.222 ± 0.072 | 38.472 ± 1.293 | 53.949 ± 2.356 | 27.791 ± 1.530 | 140.708 ± 4.439 | 81.714 ± 4.869 | 37.350 ± 2.379 |
| WT-baseline | 1.456 ± 0.050 | 22.009 ± 1.225 | 61.788 ± 2.174 | 32.708 ± 1.578 | 102.919 ± 3.971 | 56.517 ± 2.547 | 21.629 ± 1.648 |
| F11-Het-baseline | 1.397 ± 0.022 | 21.988 ± 0.658 | 59.429 ± 1.361 | 30.888 ± 0.982 | 103.055 ± 2.030 | 54.450 ± 4.070 | 22.037 ± 1.723 |
| WT-HFpEF | 2.268 ± 0.181 | 39.647 ± 1.506 | 56.610 ± 1.843 | 29.325 ± 1.238 | 115.881 ± 4.280 | 70.699 ± 1.607 | 30.731 ± 1.620 |
| 11-Het-HFpEF | 3.083 ± 0.260 | 48.331 ± 2.753 | 60.061 ± 2.530 | 31.830 ± 1.770 | 140.062 ± 5.694 | 78.215 ± 2.579 | 31.067 ± 1.673 |

d = diastolic;
s = systolic

### Intraperitoneal Glucose Tolerance Test

Intraperitoneal glucose tolerance tests (i.p. GTT) were performed by injecting glucose (2 g/kg body weight in sterile saline) after 16-hour fasting (overnight). Tail blood glucose levels were measured with a glucometer before (0 min) and at 15, 30, 60, and 120 min after glucose administration.

### Body Mass Measurement

Total body mass (fat mass and lean mass) was measured by magnetic resonance imaging (MRI) using Bruker Minispec according to manufacturer's instructions. Anesthesia was not required and mice were returned to original cages immediately after test.

### Exercise Exhaustion Test

After three days of acclimatization to treadmill, exercise exhaustion test was performed in the mice. Mice ran on the treadmill (20°) at warming-up speed of 5 m/min and the speed was increased gradually to 18 m/min. The speed was kept at 18 m/min until the mice were exhausted. Exhaustion was defined as the inability of the mice to return to running within 10 seconds of direct contact with an electric-stimulus grid. Running time was recorded and running distance was calculated. Anesthesia was not required and mice were returned to original cages immediately after test.

### Complete Blood Cell Profiling

Blood was collected with a BD Microtainer (Tubes with K2EDTA, #365974) from the retro-orbital plexus under isoflurane anesthesia. Complete blood cell profiling was assessed with the Heska (Loveland, CO) HemaTrue Veterinary Hematology Analyzer.

### Plasma and Heart Lipids Measurement

After 4-hour fasting, blood was collected from retro-orbital plexus under isoflurane in a BD Microtainer (Tubes with K2EDTA, #365974) and plasma was collected from centrifugation at 10,000 rpm for 5 min at 4° C. Total plasma cholesterol concentration was determined by enzymatic procedures in 96-well plates using a Biomek 1000 Automated Laboratory Workstation (Beckman). Plasma insulin was measured with Alpco Diagnostics Mouse ultrasensitive ELISA kit and glucose was measured with Stanbio Laboratory procedure (#1070). Free fatty acids were measured with Wako Diagnostics HR series NEFA-HR (2) kit. Samples were measured at a wavelength of 490 nm with a Vmax Microplate Reader (Molecular Devices, Inc.). Each sample was measured in triplicate.

### Western Blotting

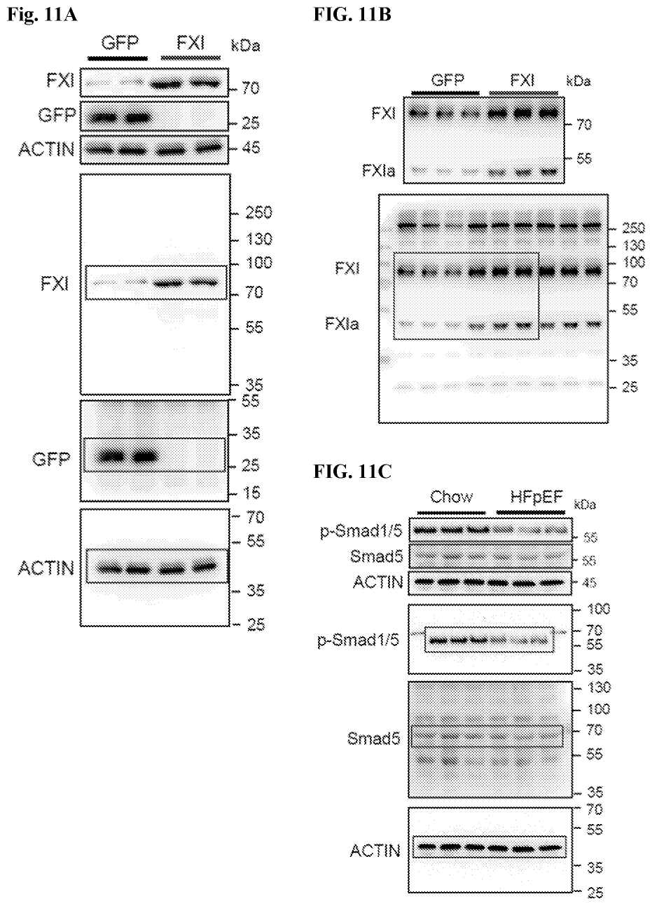

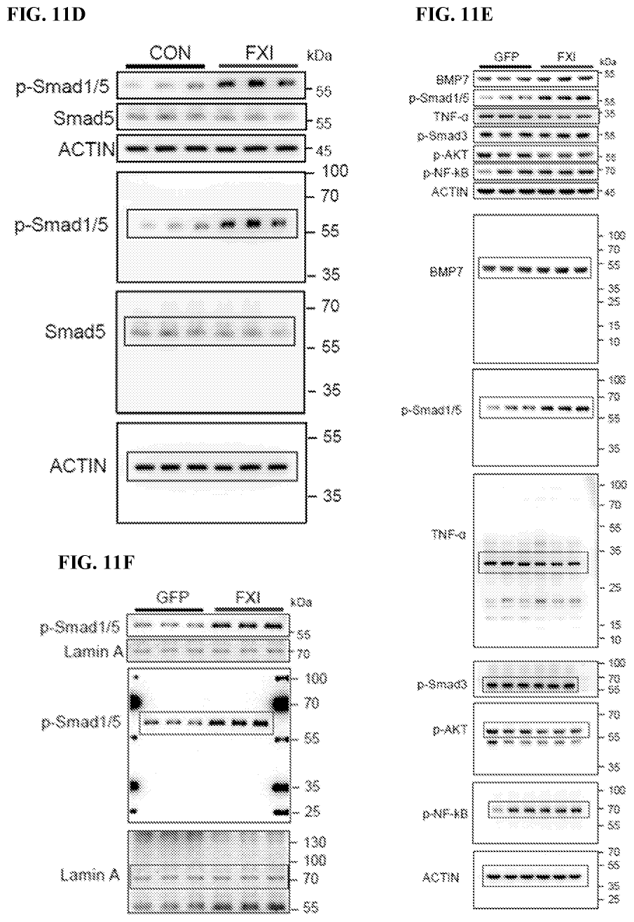

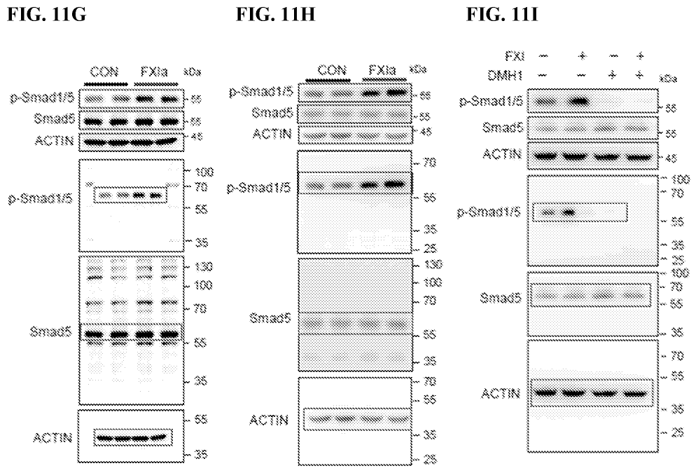

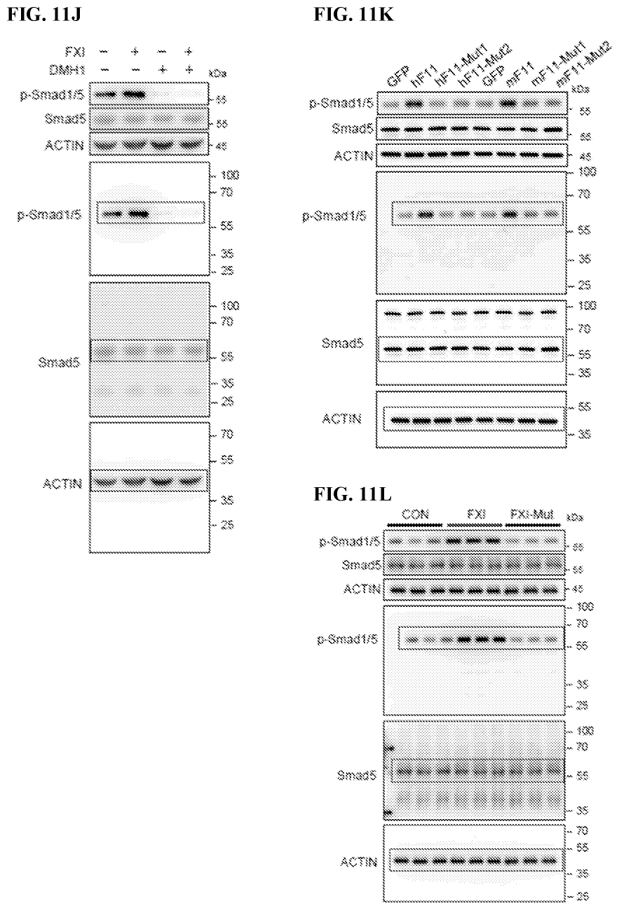

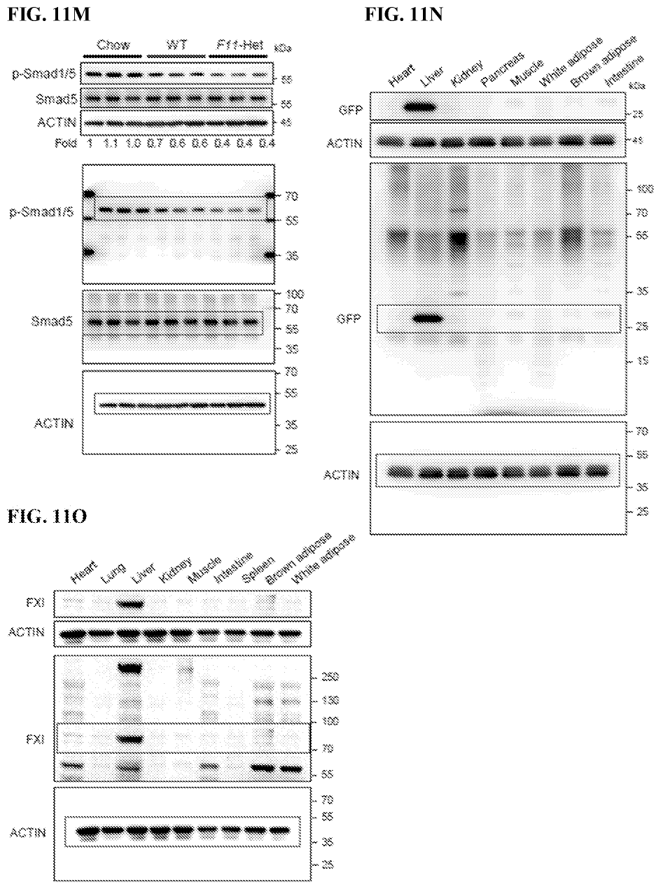

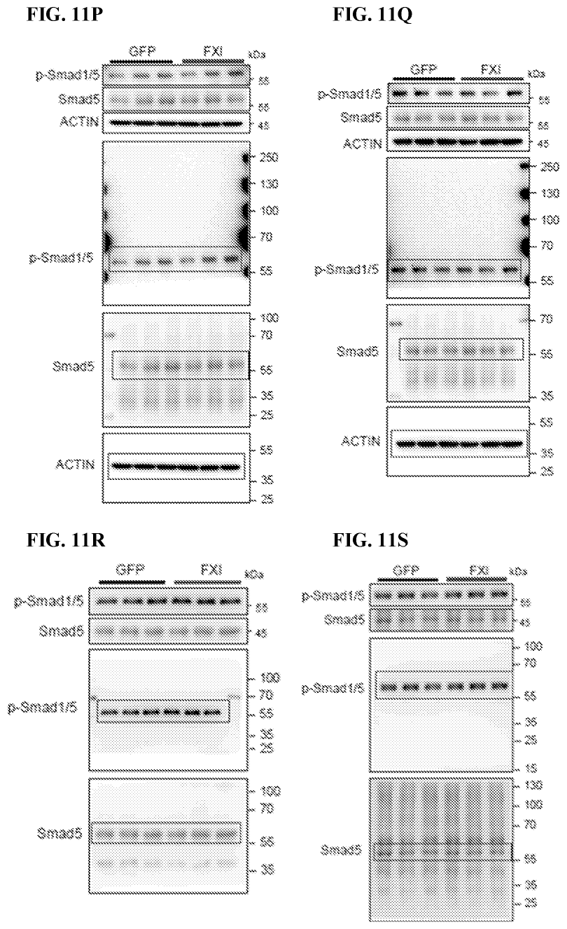

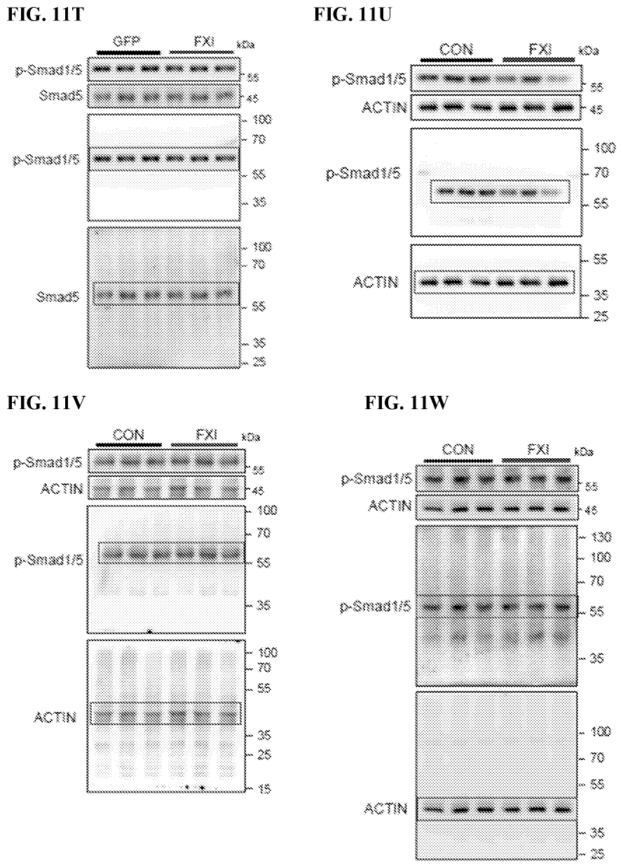

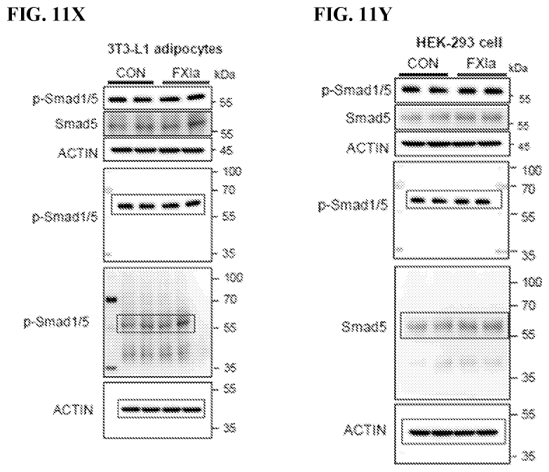

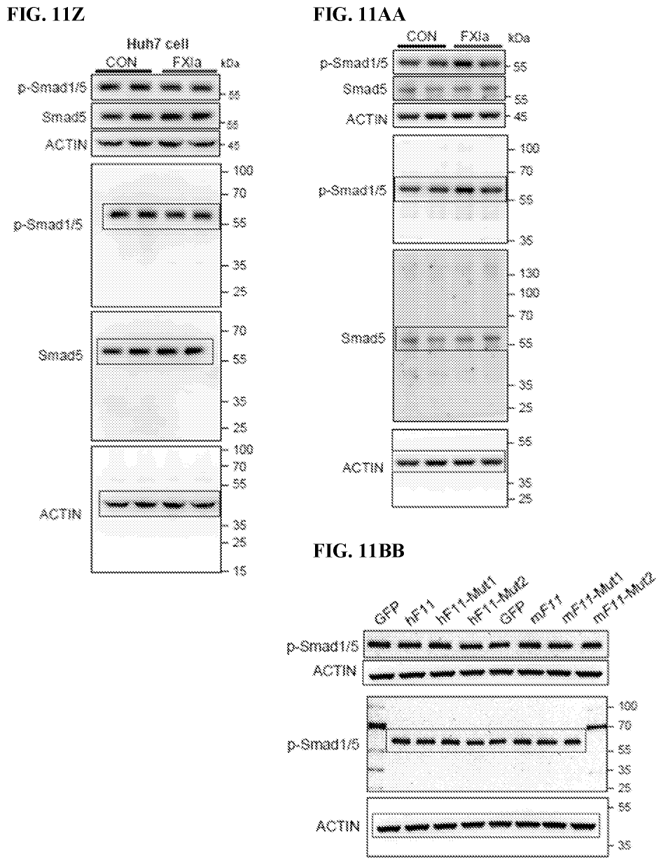

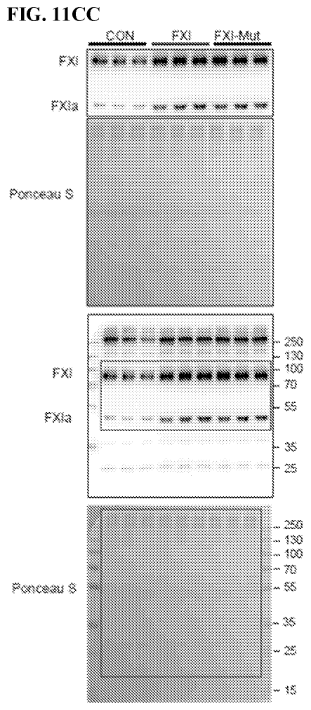

Cells and tissues were lysed in Whole Cell Extraction buffer (WCE) containing 62.5 mM Tris-HCl (pH 6.8), 2% (wt/v) sodium dodecylsulfate, and 10% glycerol. Protein content was measured using a BCA protein assay kit (Thermo Fisher #23235). Total protein concentration was normalized to 1-3 μg/μL, and samples were then denatured in 4×LDS loading buffer (Thermo Fisher #NP0007) at 99° C. for 5 min. Plasma samples were 1:10 diluted with PBS and denatured in 4×LDS loading buffer at 99° C. for 5 min. Samples were then loaded at 10 μL/well into 4%-12% Bis-Tris gels (Invitrogen) and separated out at 80 volts for 2 hours. Protein was then transferred to PVDF membranes (Immobilon) for 2 hours at 100 volts. Following transfer, membranes were stained with Ponceau S (Tocris Cat #5225) and then blocked in 5% skim milk (Gibco) in TBST for 1 hour at room temperature. Membranes were then placed in primary antibodies on a shaker overnight at 4° C. The following day, membranes were washed 3× in TBST then placed in secondary antibodies (1:10000) for 1 hour at room temperature. Blots were then washed 3× in TBST and placed Amersham ECL detection solution (GE health sciences). Blots were imaged using Amersham ImageQuant 800 biomolecular imager and bands were quantified using ImageJ Software. Full scans of cropped representative blots are shown in FIGS. 11A-11BB. The following antibodies were used:

TABLE 2

| Antibody | Manufacturer | Catalog# | dilution |
|---|---|---|---|
| p-Smad1/5 (Ser463/465) | Cell Signaling | 9516S | 1:1000 |
| Smad5 | Cell Signaling | 12534S | 1:1000 |
| p-Smad3(Ser423/425) | Cell Signaling | 9520S | 1:1000 |
| TNF-α | Santa Cruz Biotech | sc-52746 | 1:1000 |
| p-AKT(Thr308) | Cell Signaling | 13038S | 1:1000 |
| p-NF-κB(Ser536) | Cell Signaling | 3033S | 1:1000 |
| BMP7 | Santa Cruz Biotech | sc-53917 | 1:1000 |
| ACTIN | Cell Signaling | 4967S | 1:2000 |
| Lamin A | Santa Cruz Biotech | sc-518013 | 1:500 |
| GFP | Cell Signaling | 2955S | 1:3000 |
| FXI | Thermo Fisher | BS-10336R | 1:1000 |
| Rabbit secondary antibody | Cell Signaling | 7074P2 | 1:10000 |
| Mouse secondary antibody | Cell Signaling | 7076S | 1:10000 |

### Enzyme-Linked Immunosorbent Assay (ELISA)

Plasma FXI protein levels were determined by Mouse Coagulation factor XI ELISA Kit (Signalway Antibody #EK2353) according to manufacturer's instructions. Briefly, blood was collected in a BD Microtainer (Tubes with K2EDTA, #365974) and plasma was collected from centrifugation at 10,000 rpm for 5 min at 4° C. Standards (0 to 20 ng/mL concentration in 100 μL) or plasma samples (1:50 dilution in 100 μL) were added to the plate pre-coated with an antibody specific to FXI. After 2 hours of incubation at 37° C., biotin-conjugated polyclonal antibody for FXI was added to each well and incubated for 1 hour. Then avidin conjugated Horseradish Peroxidase (HRP) was added to each well and incubated for 1 hour. After wash, a TMB substrate solution (ThermoFisher, Cat #N301) was added. The reaction was terminated by adding a sulphuric acid solution and the color change was measured with a microplate reader at 450 nm. The concentration of FXI was determined by comparing the O.D. of the samples to the standard curve.

Plasma samples were collected for cytokine ELISA analysis following a standard protocol from the BD Biosciences. The coating and biotinylated antibodies for the detection of mouse IFN-γ (coating antibody, Cat #554424; biotinylated detection antibody, Cat #554426) were purchased from BD Biosciences. The coating and biotinylated antibodies for the detection of mouse IL-1b (coating antibody, Cat #554424; biotinylated detection antibody, Cat #554426) were purchased from Invitrogen. IL-6 coating antibody (Cat #504502) and biotinylated detection antibody (Cat #504602) were purchased from Biolegend. The streptavidin-HRP conjugate (Cat #18410051) was purchased from Invitrogen. The absorbance at 450 nm was measured using an Infinite M1000 microplate reader (Tecan).

### Thrombin-Antithrombin Complexes Measurement

TAT Complexes in mouse plasma were measured with Mouse Thrombin-Antithrombin Complexes ELISA Kit (TAT) (Abcam, Cat #ab137994) according to manufacturer's instructions. Briefly, an antibody specific for TAT complexes was precoated onto the plate and blocked. Standards or plasma samples (1:100 diluted) were added to the wells (50 μL/well) and incubated for 2 hours at room temperature. Then a TAT Complexes specific biotinylated detection antibody was added to each well and incubated for 2 hours at room temperature. After wash, streptavidin-peroxidase conjugate was added to each well and incubated for 30 min. After wash, TMB was added to visualize streptavidin-peroxidase enzymatic reaction (blue) and acidic stop solution was then added to stop the reaction (color changed to yellow). The density of coloration was measured with a microplate reader at 450 nm and was proportional to the amount of TAT Complexes.

### Multiplex-Immunohistochemistry (IHC)

Inflammatory cells in the heart tissue were stained with IHC using the manual Opal 7-Color IHC Kit (NEL811001KT) with modification. After sacrifice, mouse hearts were prepared with formalin-fixed, paraffin-embedded (FFPE) techniques. Slide was dewaxed with xylene (3×10 min) and rehydrate through a graded series of ethanol solutions: (100% 1×5 min; 95% 1×5 min; and 70% 1×2 min) and washed in distilled water (1×2 min) and TBST (1×2 min). Then slides were placed in a plastic jar with AR buffer. Slides were boiled with microwave for 45 sec at 100% power and an additional 15 min at 20% power. Then slides were washed in distilled water (1×2 min) and TBST (1×2 min). The tissue sections were covered with blocking buffer (PerkinElmer antibody diluent buffer #ARD1001EA) and incubated in a humidified chamber for 10 min at room temperature. Then primary antibodies were applied overnight or at room temperature for 1 hr according to antibody sensitivity. After incubation, slides were washed with TBST for 3 times and incubated with secondary antibodies for 30 min at room temperature. Slides were washed 3 times with TBST and incubated with opal Fluorophore Working Solution (1:50 dilution) for 10 min to amplify the signals. After signal amplification, slides were washed 3 times with TBST, boiled by microwave in AR buffer and washed with distilled water and TBST to strip the antibody complex. The steps of blocking and incubating with the first antibody and secondary antibody were repeated for each antibody. After incubation with the antibodies, slides were incubated with DAPI (Cat #P36931, 1:2000 dilution) and mounted with mounting medium. The following first antibodies were used:

TABLE 3

| Primary Antibody | Manufacturer | Catalog# | dilution |
|---|---|---|---|
| F4/80 | SEROTEC | MCA497B | 1:100 |
| CD3 | DAKO | A0452 | 1:200 |
| Ly6C | Abcam | ab15627 | 1:100 |
| ly6G | BD | 551459 | 1:500 |

### Masson's Trichrome Staining

Fibrosis in heart tissue was visualized by Masson's Trichrome staining of formalin-fixed, paraffin-embedded sections. Collagen and fiber were stained differentially when treated with Biebrich Scarlet-Acid Fuchsin, phosphomolybdic/phosphotungstic acid (PTA/PMA) and Aniline Blue. The collagen fibers were stained blue and the nuclei were stained black and the background was red. Fibrosis area was visualized with a Aperio ImageScope viewing software (Leica Biosystem) and quantified using ImageJ software version 2.0.

### RNA Extraction and Quantitative RT-PCR

Total RNA was isolated using Trizol reagent (Invitrogen) and followed by DNase (Ambion) treatment. cDNA was synthesized using the iScript cDNA Synthesis Kit (Bio-Rad) and cDNA samples were diluted 1:10 with ddH₂O. Annealing temperatures for each pair of primers were optimized by temperature gradient PCR. Quantitative real-time PCR was performed using iQ SYBR Green Supermix and the iCycler Real-time PCR Detection System (Bio-Rad). Each target mRNA was compared to 18S rRNA or actin, and the fold change of target mRNA expression was calculated based on threshold cycle (Ct), where ΔCt=Cttarget−Ct18S and Δ (ΔCt)=ΔCt Control−ΔCt Indicated condition. All Sequences to qPCR primers are listed below:

TABLE 4

| | | | SEQ |
|---|---|---|---|
| | | | ID |
| Species | Gene | Sequence (5'-3') | NO |
| Mouse | F11-F | GCCAATGGTAAACATGACAGGC | 1 |
| Mouse | F11-R | TGTCTAGGTTCACGTACACATCT | 2 |
| Mouse | Col5a3-F | CGGGGTACTCCTGGTCCTAC | 3 |
| Mouse | Col5a3-R | GCATCCCTACTTCCCCCTTG | 4 |
| Mouse | Col5a1-F | CTTCGCCGCTACTCCTGTTC | 5 |
| Mouse | Col5a1-R | CCCTGAGGGCAAATTGTGAAAA | 6 |
| Mouse | Adam19-F | TCAGTGGCGGACTTCAGAAAG | 7 |
| Mouse | Adam19-R | GCAAAAAGGTGCTCGTTCTTC | 8 |
| Mouse | IL1b-F | GAAATGCCACCTTTTGACAGTG | 9 |
| Mouse | IL1b-R | TGGATGCTCTCATCAGGACAG | 10 |
| Mouse | IL6-F | AGTTGCCTTCTTGGGACTGA | 11 |
| Mouse | IL6-R | TCCACGATTTCCCAGAGAAC | 12 |
| Mouse | Tnfa-F | CCTGTAGCCCACGTCGTAG | 13 |
| Mouse | Tnfa-R | GGGAGTAGACAAGGTACAACCC | 14 |
| Mouse | Bmp7-F | CCTGTCCATCTTAGGGTTGCC | 15 |
| Mouse | Bmp7-R | GGCCTTGTAGGGGTAGGAGA | 16 |
| Mouse | Actin-F | GGCTGTATTCCCCTCCATCG | 17 |
| Mouse | Actin-R | CCAGTTGGTAACAATGCCATGT | 18 |
| Rat | Nppa-F | TTCGGTACCGGAAGCTGTTG | 19 |
| Rat | Nppa-R | CTGGACTGGGGAAGTCAACC | 20 |
| Rat | Nppb-F | GTGCTGCCCCAGATGATTCT | 21 |
| Rat | Nppb-R | GGCGCTGTCTTGAGACCTAA | 22 |
| Rat | Col5a3-F | CGGGGTGTGCCTGGTCCTAC | 23 |
| Rat | Col5a3-R | GCATCCCTACTTCCCCCTTG | 24 |
| Rat | Adam19-F | TCAGTGGAGGACTTCAGAAAG | 25 |
| Rat | Adam19-R | GCAAAAAGGTGCTCGTTCTTC | 26 |
| Rat | 18s-F | GACAGGATTGACAGATTGATAGC | 27 |
| Rat | 18s-R | AGTCTCGTTCGTTATCGGAAT | 28 |
| Rat | Actin-F | GGCCGTCTTCCCCTCCATCG | 29 |
| Rat | Actin-R | CCAGTTGGTGACAATGCCGTGT | 30 |
| Human | F11-F | GGAGGGGACATTACTACGGTC | 31 |
| Human | F11-R | ATTCCGCCGTGAAAGTGAAGA | 32 |
| Human | COL5A3-F | GTGGCCGTCAGCATAGATGG | 33 |
| Human | COL5A3-R | TGAATGTCTCCCTCGAAAGTCTT | 34 |
| Human | ADAM19-F | ACCCTCAAACCACCACACG | 35 |
| Human | ADAM19-R | GCTCACCGTAATCAGTCCTCTA | 36 |
| Human | NPPA-F | CAACGCAGACCTGATGGATTT | 37 |
| Human | NPPA-R | AGCCCCCGCTTCTTCATTC | 38 |
| Human | NPPB-F | TGGAAACGTCCGGGTTACAG | 39 |
| Human | NPPB-R | CTGATCCGGTCCATCTTCCT | 40 |
| Human | Actin-F | CATGTACGTTGCTATCCAGGC | 41 |
| Human | Actin-R | CTCCTTAATGTCACGCACGAT | 42 |

### Library and RNA-Seq

Total RNA was extracted with miRNeasy Mini Kit, and RNA quality was validated with BioANAlyzer (all samples had RIN>8). RNA libraries were prepared with Nugen Universal mRNA-Seq kit. Sequencing was performed at the UCLA Technology Center for Genomics & Bioinformatics (TCGB). Gene expression was analyzed using 2×50 NovaSeq S2 and reads were quantified against the GRCm38.p6 mouse reference transcriptome (Ensembl release 97) using kallisto version 0.46.0. Differential expression analysis was performed with DESeq2 1.28.1.

### Association Analysis

Liver-heart cross tissue biweight midcorrelation coefficients and p-values were calculated using R package weighted gene co-expression network analysis (WGCNA)⁶. Genome-wide association of clinical traits and liver and heart expression data was performed using FaST-LMM³¹. Significance threshold of (3.46×10-⁶) was determined through permutation and modeling. Linkage disequilibrium (LD) was determined by calculated pairwise r² SNP correlations for each chromosome.

### Statistical Analysis

All computational procedures were carried out using R statistical software. Correlations and associated p-values were calculated with the biweight midcorrelation, which is robust to outliers and associated p-value³². Single comparisons between two groups were performed using two-tailed Student's t tests with 95% confidence intervals. Multiple comparisons were performed using an ordinary 1-way ANOVA followed by Tukey's multiple comparisons test, or using a 2-way ANOVA followed by Sidak's multiple comparisons test. Values were considered significant at p<0.05. Unless otherwise noted, values presented are expressed as means±SEM.

### Example 2: Identification and Charactization of Secreted Proteins that Mediate Communication Between the Liver and Heart

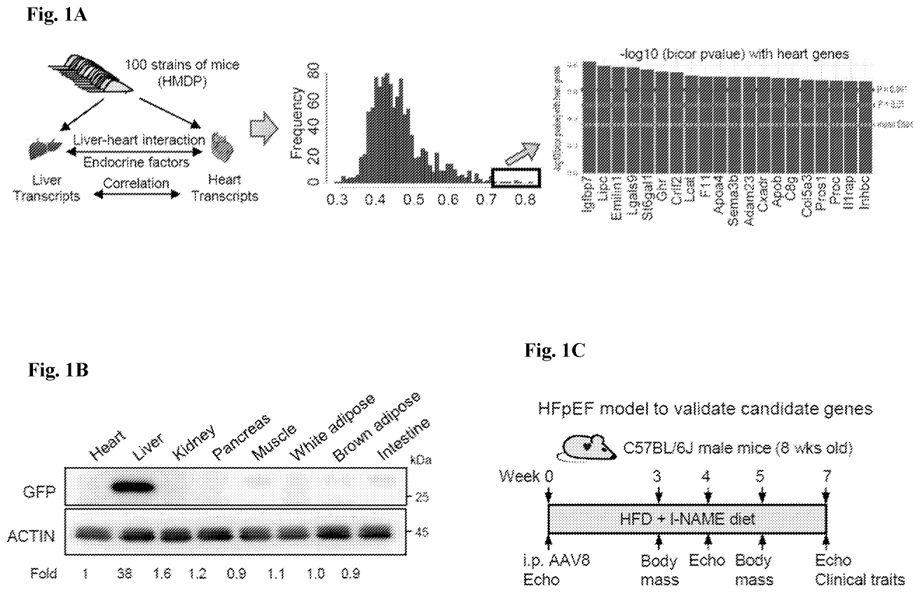

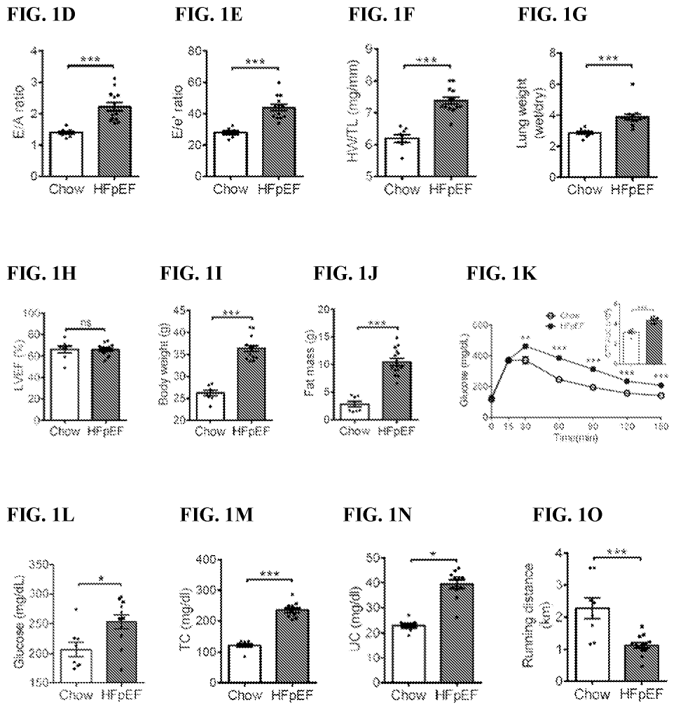

Tissue-tissue crosstalk by endocrine factors, including secreted proteins¹, is a vital mechanism to maintain proper physiologic homeostasis. The heart and the liver display multifaceted interactions² and in clinical practice it is common to observe heart diseases affecting the liver and visa versa.³ For instance, non-alcoholic fatty liver disease (NAFLD) increases the risk for heart failure with diastolic and systolic dysfunction.^{4,5} It was hypothesized that novel secreted proteins may mediate communication between liver and heart. To identify such factors, a recently developed bioinformatics approach was employed that uses natural variation in populations to identify novel endocrine circuits.⁶ The Hybrid Mouse Diversity Panel (HMDP),⁷ a resource consisting of about 100 diverse inbred strains of mice, was used as the population. Global transcriptomic data from the heart and the liver were generated across all 100 inbred strains and used to the detect correlation structure between the secreted proteins (from the liver) and their downstream effects in the heart (FIG. 1A). By assessing the strength of cross-tissue predictions, a list was generated of potential liver-heart mediators (FIG. 1A and Table 5). The top-ranked candidates include Igfbp7, Lipc, Emilin1, Lgals9, St6gal1, Ghr, Crlf2, Lcat, and F11. This list revealed several previously described mediators with consistent functions. For instance, insulin-like growth factor-binding protein-7 (Igfbp7) has been reported to be significantly correlated with diastolic function in heart failure with reduced ejection fraction (HFrEF) and heart failure with preserved ejection fraction (HFpEF) patients.⁸

TABLE 5
Top 100 liver-heart mediators

| Score | gene_symbol |
|---|---|
| 0.826771 | Igfbp7 |
| 0.794016 | Lipc |
| 0.786771 | Emilin1 |
| 0.782647 | Lgals9 |
| 0.767882 | St6gal1 |
| 0.753807 | Ghr |
| 0.748098 | Crlf2 |
| 0.72286 | Lcat |
| 0.717331 | F11 |
| 0.715282 | Apoa4 |
| 0.714212 | Sema3b |
| 0.712534 | Adam23 |
| 0.711667 | Cxadr |
| 0.705562 | Apob |
| 0.703338 | C8g |
| 0.690702 | Col5a3 |
| 0.688219 | Pros1 |
| 0.686463 | Proc |
| 0.681954 | Il1rap |
| 0.679971 | Inhbc |
| 0.672619 | Ccdc80 |
| 0.672231 | Dpp4 |
| 0.671719 | Cd9 |
| 0.670576 | Hgfac |
| 0.670049 | Tor2a |
| 0.670007 | Ndfip1 |
| 0.663397 | Kdr |
| 0.658814 | Serping1 |
| 0.657397 | C1qtnf1 |
| 0.657162 | Apof |
| 0.655221 | Sec63 |
| 0.654943 | Itm2b |
| 0.65361 | Igfbp1 |
| 0.653126 | Sod3 |
| 0.651735 | Apoc2 |
| 0.650295 | Bgn |
| 0.64993 | Wnt5b |
| 0.649266 | Pon1 |
| 0.641637 | F13b |
| 0.641263 | Lifr |
| 0.639418 | Crtap |
| 0.639215 | Lgals1 |
| 0.636201 | Lgals3bp |
| 0.635223 | Leap2 |
| 0.634324 | Bmp7 |
| 0.633231 | Clu |
| 0.629767 | Apoa1 |
| 0.62958 | Enpp5 |
| 0.626289 | Igfals |
| 0.626022 | Abhd15 |
| 0.620676 | Cgref1 |
| 0.619507 | Oit3 |
| 0.618952 | Fuca2 |
| 0.61837 | Mug1 |
| 0.616026 | Entpd5 |
| 0.615847 | Prdx4 |
| 0.615637 | Clca1 |
| 0.614844 | Fgf1 |
| 0.614277 | Tuft1 |
| 0.614131 | Ecm1 |
| 0.612692 | Zp3 |
| 0.609909 | Apom |
| 0.609394 | Angptl6 |
| 0.608357 | Timp4 |
| 0.608219 | Itih1 |
| 0.607719 | Dnase1 |
| 0.607034 | Col18a1 |
| 0.606395 | Habp2 |
| 0.606305 | Cfp |
| 0.605259 | Tnfsf14 |
| 0.604951 | Ngrn |
| 0.603275 | Gpx3 |
| 0.602284 | Mup4 |
| 0.600921 | Lama4 |
| 0.600211 | Cxcl16 |
| 0.599385 | Vegfc |
| 0.59801 | Obp2a |
| 0.597661 | Xdh |
| 0.596987 | Serpinf1 |
| 0.596096 | Hmgb1 |
| 0.594009 | Cant1 |
| 0.593987 | Lect2 |
| 0.591955 | Creg1 |
| 0.591822 | Serpinc1 |
| 0.591673 | Serpinf2 |
| 0.591275 | Gfod2 |
| 0.590747 | Ctsb |
| 0.590476 | Calu |
| 0.590353 | Pglyrp2 |
| 0.589324 | Serpina10 |
| 0.588891 | Spp2 |
| 0.587791 | Alb |
| 0.586507 | Srgn |
| 0.583023 | Pf4 |
| 0.582114 | Gdf2 |
| 0.581088 | Ctsd |
| 0.580895 | Timp2 |
| 0.580547 | Mbl1 |
| 0.579637 | B2m |
| 0.579462 | Col6a3 |

The roles of several of these factors were validated and characterized in a mouse model of HFpEF. It was hypothesized that an effect would more likely be seen if the heart was stressed. Based on tissue distribution and functional annotation, Hgfac, C8g, and F11 were selected for analysis (FIG. 1A). These genes were overexpressed individually in the livers of C57BL/6J male mice with an adeno-associated virus serotype 8 (AAV8) vector carrying a nucleic acid sequence encoding HGFAC, C8G, or FXI proteins or a nucleic acid encoding a GFP control. Expression of the transgenes were under the control of the liver-specific thyroid hormone-binding globulin (TBG) promoter (FIG. 1B). After AAV8 injection, mice were subjected to a “two-hit” HFpEF model induced by a combination of high-fat diet (HFD) and inhibition of nitric oxide synthase using Nω-nitrol-arginine methyl ester (1-NAME)¹² and then cardiac functions were assessed (FIG. 1C). After 7 weeks of HFD+1-NAME feeding, mice developed heart failure phenotypes that recapitulate clinical symptoms of HFpEF, such as diastolic dysfunction (increased E/A ratio (the ratio of peak velocity blood flow from left ventricular relaxation in early diastole (the E wave) to peak velocity flow in late diastole caused by atrial contraction (the A wave)), E/e′ ratio (ratio of mitral peak velocity of early filling (E) to early diastolic mitral annular velocity (e′)), left ventricular (LV) mass, heart weight and lung weight), metabolic disorders (increased body weight, fat mass, plasma lipids, and glucose intolerance), exercise intolerance (reduced running distance) as well as preserved ejection fraction (LVEF) (FIGS. 1D-10).

### Example 3: a Potential Role for FXI in HFpEF

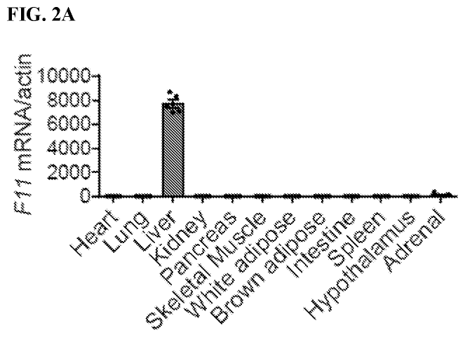

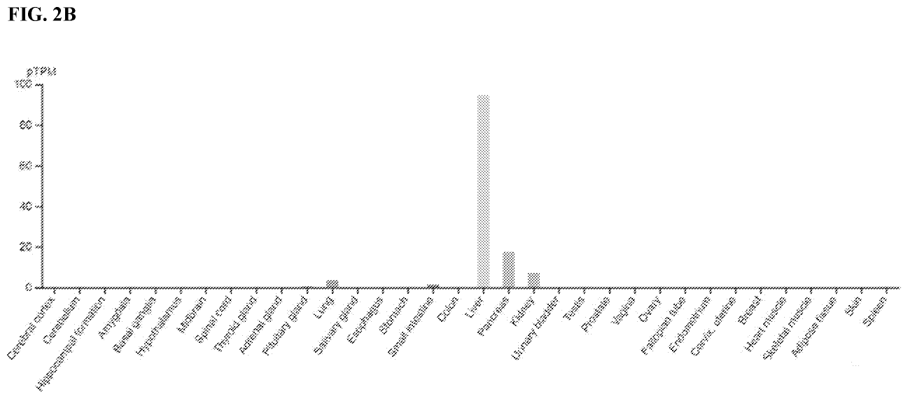

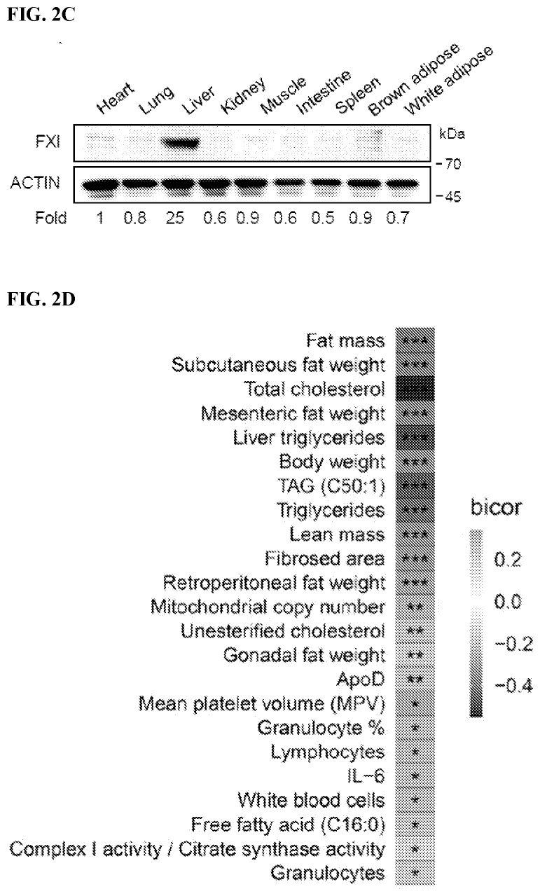

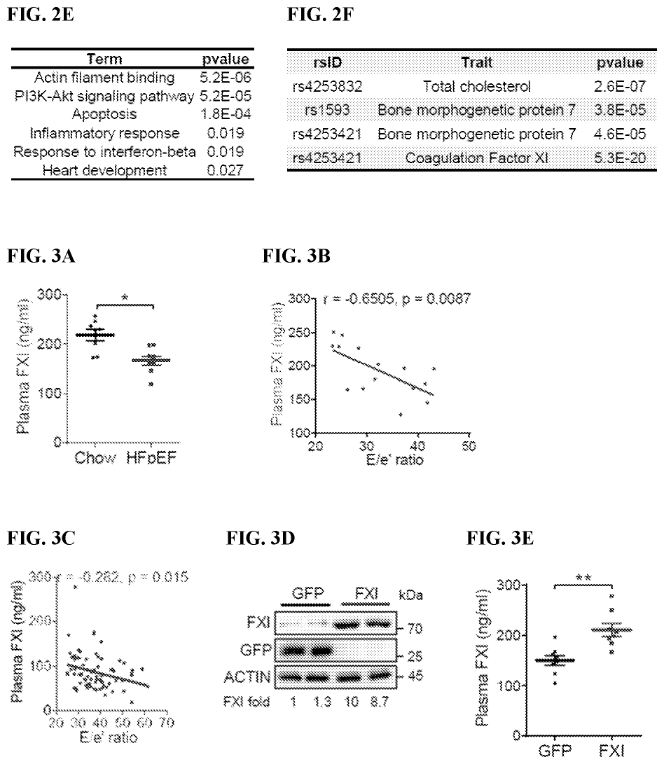

Overexpressed liver-derived hepatocyte growth factor activator (HGFAC) increased LV mass and complement C8 gamma chain (C8G) decreased heart weight in the model of HFpEF (data not shown). However, focus was placed on Coagulation Factor 11 (FXI), since it affected several HFpEF traits, including diastolic function. It acts downstream of Factor X11^{13, 14} and triggers the middle phase of the intrinsic pathway of blood coagulation by activating Factor IX. Like HGFAC and C8G, FXI is also exclusively expressed in the liver (FIGS. 2A-2C). Based on association with heart transcript levels in the HMDP, it was predicted that FXI would be strongly correlated with critical pathways in the heart and a number of clinical traits that were important for HFpEF (FIGS. 2D and 2E). In addition, human genome-wide association studies (GWAS) revealed that genetic loci encompassing the F11 gene were associated with the total cholesterol and bone morphogenetic protein 7 (BMP7) levels (FIG. 2F and Table 6). These data indicates a potential role of FXI in heart failure.

TABLE 6
F11 mapping to loci in human GWAS

| Gene | rsID | Trait | pvalue | Direction | EA | NEA |
|---|---|---|---|---|---|---|
| F11 | rs1593 | Interleukin-2 | 1.91E−05 | + | A | T |
| F11 | rs1593 | Pulmonary heart disease | 3.41E−05 | − | T | A |
| F11 | rs1593 | Bone morphogenetic protein 7 | 3.8E−05 | + | A | T |
| F11 | rs1593 | Pulmonary heart disease, diseases of | 8.99E−05 | − | T | A |
| F11 | rs1593 | Mitochondrial ubiquitin ligase activator of | 0.00057 | − | A | T |
| F11 | rs1593 | T-cell surface glycoprotein CD3 epsilon | 0.00069 | − | A | T |
| F11 | rs203691 | Interleukin-13 receptor subunit alpha-1 | 5.37E−07 | + | C | T |
| F11 | rs203691 | Free cholesterol to total lipids ratio in IDL | 9.7E−05 | + | C | T |
| F11 | rs203691 | 72 kDa type IV collagenase | 0.00012 | + | C | T |
| F11 | rs203691 | Interleukin-2 | 0.00021 | + | C | T |
| F11 | rs203691 | Coronary heart disease | 0.00028 | − | C | T |
| F11 | rs203691 | Coronary heart disease | 0.00028 | − | C | T |
| F11 | rs203691 | Interleukin-22 receptor subunit alpha-1 | 0.00038 | − | C | T |
| F11 | rs203691 | Interleukin-16 levels | 0.00063 | − | C | T |
| F11 | rs203691 | Immunoglobulin superfamily member 8 | 0.00083 | − | C | T |
| F11 | rs228925 | Pulmonary heart disease | 2.49E−11 | + | T | C |
| F11 | rs228925 | Pulmonary heart disease, diseases of | 1.51E−09 | + | T | C |
| F11 | rs228925 | Tumor necrosis factor receptor superfamily | 7.76E−07 | − | T | C |
| F11 | rs228925 | 72 kDa type IV collagenase | 6.61E−06 | + | T | C |
| F11 | rs228925 | Fructose-2,6-bisphosphatase TIGAR | 9.55E−05 | − | T | C |
| F11 | rs228925 | Interleukin-13 receptor subunit alpha-1 | 0.00014 | + | T | C |
| F11 | rs228925 | Whole body water mass | 0.00019 | + | T | C |
| F11 | rs228925 | Whole body fat-free mass | 0.00025 | + | T | C |
| F11 | rs228925 | Insulin-like growth factor-binding protein 7 | 0.00027 | + | T | C |
| F11 | rs228925 | Lipoprotein A | 0.00038 | − | T | C |
| F11 | rs228925 | Basal metabolic rate | 0.00063 | + | T | C |
| F11 | rs228925 | Immunoglobulin superfamily DCC subclass | 0.00069 | + | T | C |
| F11 | rs228925 | Insulin-like growth factor-binding protein 3 | 0.00072 | + | T | C |
| F11 | rs425339 | Pulmonary heart disease | 1.86E−11 | + | G | T |
| F11 | rs425339 | Pulmonary heart disease, diseases of | 1.83E−10 | + | G | T |
| F11 | rs425339 | Tumor necrosis factor receptor superfamily | 2.19E−06 | − | G | T |
| F11 | rs425339 | Congenital malformations of heart and great | 0.00085 | − | G | T |
| F11 | rs425339 | Transforming growth factor beta-1 | 0.00097 | − | G | T |
| F11 | rs425340 | Tumor necrosis factor receptor superfamily | 1.82E−07 | + | G | A |
| F11 | rs425340 | 72 kDa type IV collagenase | 0.00093 | − | G | A |
| F11 | rs425341 | Tumor necrosis factor receptor superfamily | 1.51E−06 | − | T | C |
| F11 | rs425341 | Interleukin-13 receptor subunit alpha-1 | 4.47E−06 | + | T | C |
| F11 | rs425341 | Interleukin-16 levels | 5.28E−05 | − | T | C |
| F11 | rs425341 | Free cholesterol to total lipids ratio in very | 0.00039 | + | T | C |
| F11 | rs425341 | Interleukin-2 | 0.00041 | + | T | C |
| F11 | rs425341 | Medication for cholesterol, blood pressure or | 0.00084 | − | T | C |
| F11 | rs425341 | Insulin-like growth factor-binding protein 6 | 0.00091 | − | T | C |
| F11 | rs425341 | Tumor necrosis factor receptor superfamily | 6.03E−07 | − | C | T |
| F11 | rs425341 | 72 kDa type IV collagenase | 2.88E−06 | + | C | T |
| F11 | rs425341 | Fructose-2,6-bisphosphatase TIGAR | 2.69E−05 | − | C | T |
| F11 | rs425341 | Whole body fat-free mass | 0.00012 | + | C | T |
| F11 | rs425341 | Insulin-like growth factor-binding protein 7 | 0.00020 | + | C | T |
| F11 | rs425341 | Interleukin-13 receptor subunit alpha-1 | 0.00021 | + | C | T |
| F11 | rs425341 | Basal metabolic rate | 0.00036 | + | C | T |
| F11 | rs425341 | Immunoglobulin superfamily DCC subclass | 0.00038 | + | C | T |
| F11 | rs425341 | Interleukin-6 receptor subunit beta | 0.00069 | + | C | T |
| F11 | rs425342 | Bone morphogenetic protein 7 | 4.57E−05 | + | G | A |
| F11 | rs425342 | Interleukin-2 | 6.03E−05 | + | G | A |
| F11 | rs425342 | MHC class I polypeptide-related sequence | 0.00058 | + | G | A |
| F11 | rs568105 | 72 kDa type IV collagenase | 1.55E−06 | + | T | A |
| F11 | rs568105 | Insulin-like growth factor-binding protein 7 | 7.59E−05 | + | T | A |
| F11 | rs568105 | Interleukin-6 receptor subunit beta | 0.00089 | + | T | A |

### Example 4: FXI Affects HFpEF Traits

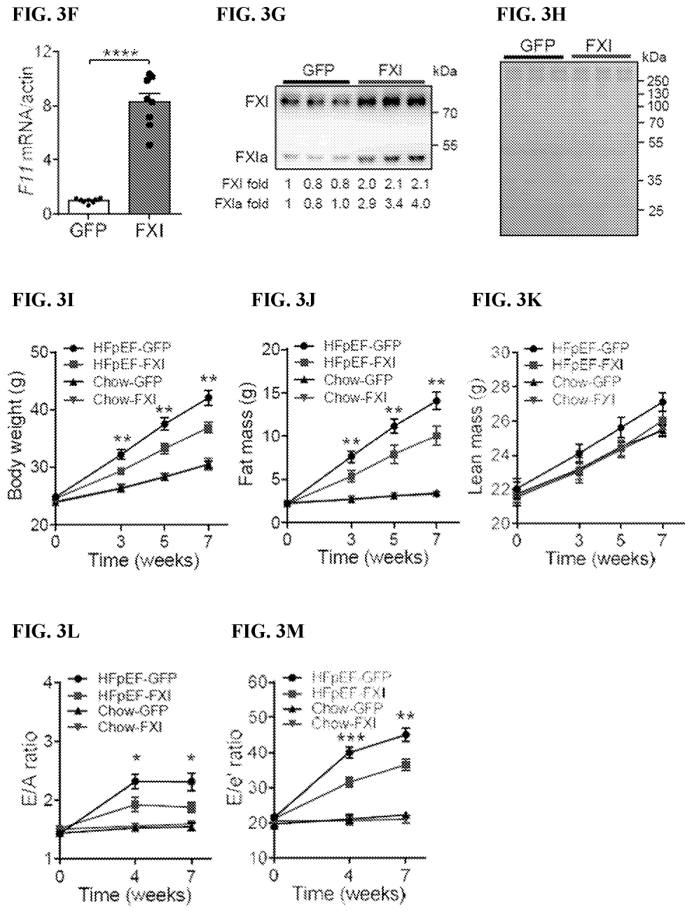

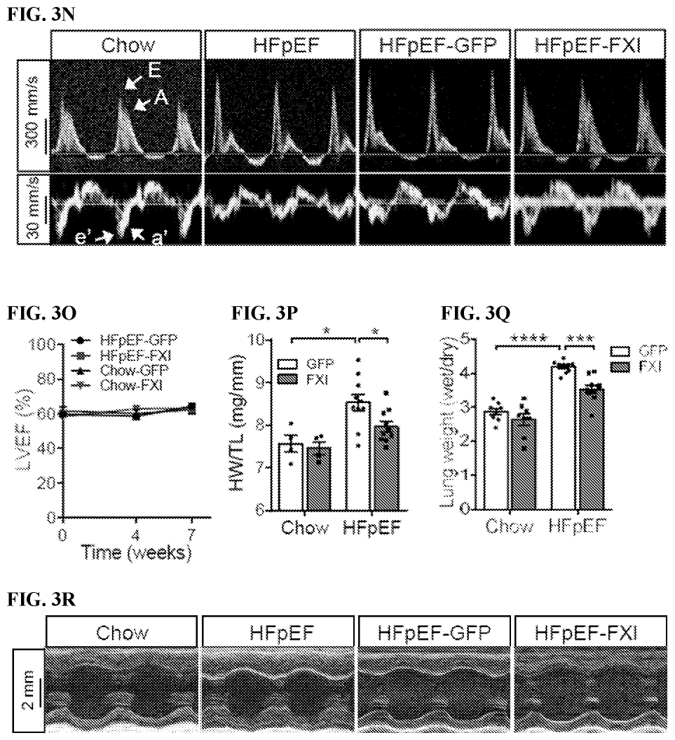

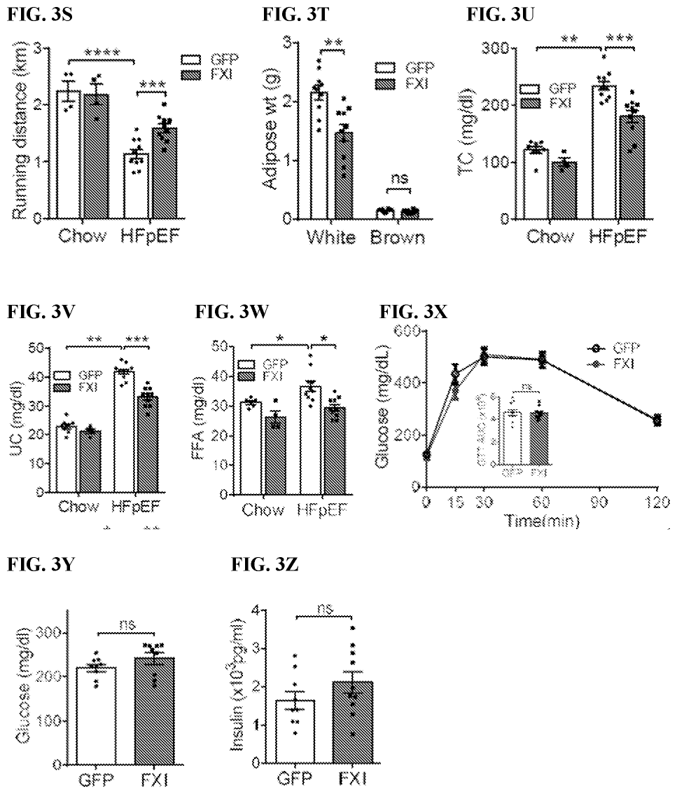

Reduced plasma FXI levels were initially observed in mice after being fed an HFD+1-NAME diet relative to chow diet, which was inversely correlated with diastolic dysfunction (FIGS. 3A and 3B). The HFpEF phenotype was then induced in 30 inbred strains of mice, a subset of HMDP, to examine the association between plasma FXI and diastolic dysfunction. FXI levels were inversely correlated with diastolic dysfunction after feeding the mice the HFpEF diet, indicating a potential impact of FXI on heart failure (FIG. 3C). The function of FXI in HFpEF model was then directly validated using overexpression. C5BL/6J male mice injected with AAV8-GFP or AAV8-F11 were then fed a chow diet or an HFD+1-NAME diet for 7 weeks (FIG. 1C). After AAV8 injection, F11 expression was elevated in the liver and FXI protein was increased in the plasma (FIGS. 3D-3G). Notably, FXI protein was not detected in the heart, supporting the concept that FXI is an endocrine factor produced by liver that affects the heart (data not shown). Mice receiving AAV8-F11 exhibited a decrease in body weight and fat mass after HFpEF compared with those receiving AAV8-GFP (FIGS. 3H-3J). Consistent with the genetic results in the HMDP population, FXI overexpression decreased E/A ratio, E/e′ ratio, heart weight, and lung weight in the HFpEF model while LVEF was preserved, indicating an improvement in diastolic function (FIGS. 3K-3Q). Running distance was also improved by FXI overexpression, indicating that FXI ameliorates exercise intolerance in HFpEF (FIG. 3R). FXI overexpression also had beneficial metabolic effects on fat mass and plasma lipid levels (FIGS. 3S-3V) but not on glucose tolerance (FIGS. 3W-3Y).

### Example 5: The Effects of FXI Overexpression on Blood Coagulation

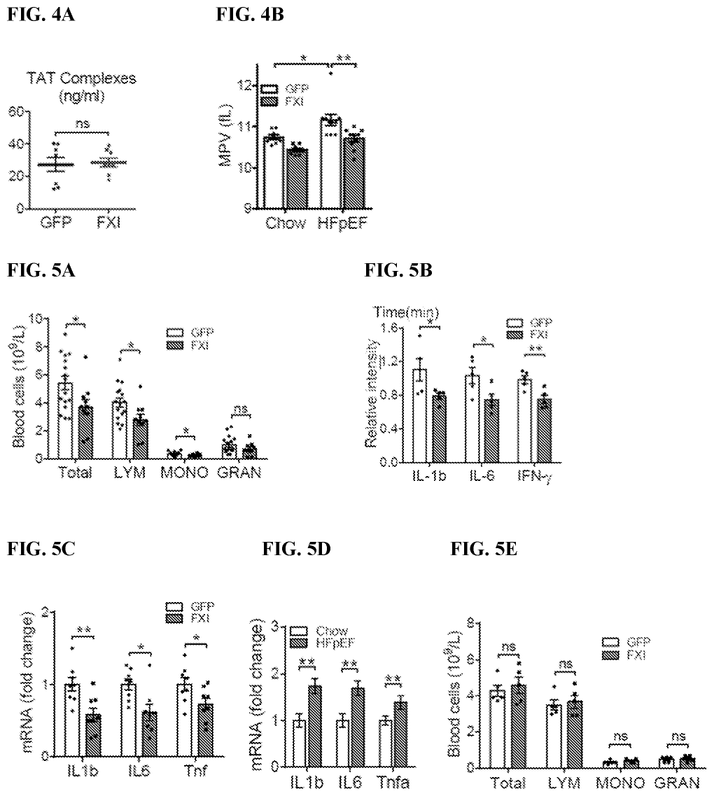

To test whether FXI overexpression affects blood coagulation, blood thrombin-antithrombin (TAT) complexes were measured in mice with GFP or FXI overexpression. TAT complexes were not significantly changed in mice receiving AAV8-F11 vs. AAV8-GFP (FIG. 4A), suggesting that coagulation system was not impacted by FXI overexpression. It has been found that mean platelet volume (MPV), reflecting the size and activity of platelets, is increased in decompensated heart failure patients and correlates with disease severity, serving as an independent predictor of six-month mortality following decompensation.¹⁵ An increase of MPV upon HFpEF development was measured and FXI overexpression reversed it (FIG. 4B).

### Example 6: The Effects of FXI on Cardiac Infiltration of Inflammatory Cells

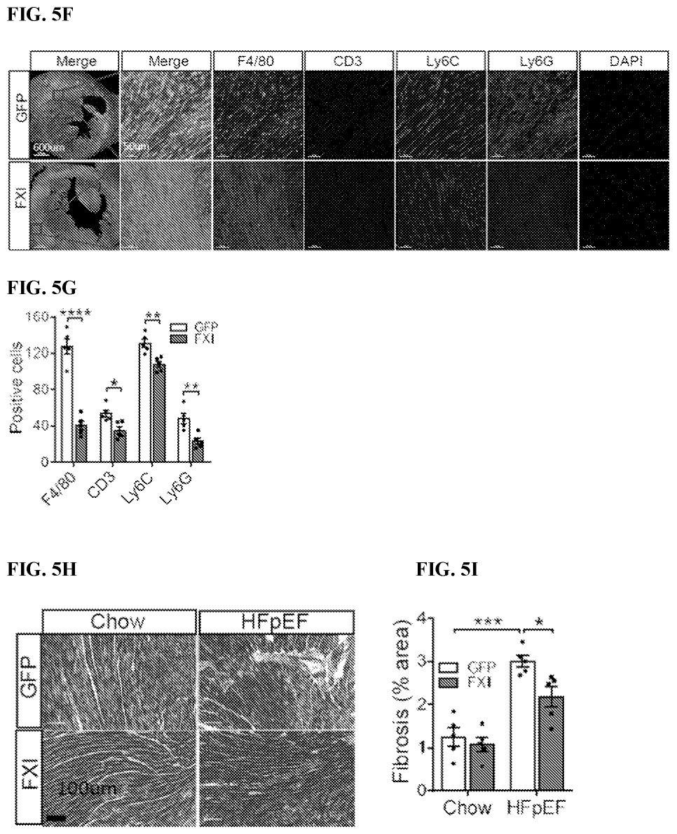

Importantly, FXI overexpression significantly reduced circulating inflammatory cells and cytokines levels in the HFpEF model (FIGS. 5A and 5B). Moreover, the expression of inflammatory genes in the heart was also reduced by FXI overexpression (FIGS. 5C and 5D). When mice were maintained on a chow diet, the number of blood immune cells was not changed by FXI overexpression (FIG. 5E). To further test whether the cardiac infiltration of inflammatory cells was attenuated by FXI, multiplex-immunohistochemistry was measured using antibodies against macrophages (F4/80), T cells (CD3), monocytes (Ly6C) and granulocytes (Ly6G). Significantly decreased inflammatory cells in heart tissue from FXI-overexpressed mice vs. GFP-overexpressed mice esd observed (FIGS. 5F and 5G), indicating that FXI overexpression reduced inflammation in heart tissue in the HFpEF model. In addition, FXI overexpression also decreased fibrosis in the heart, consistent with its predicted function (FIGS. 5H and 5H).

### Example 7

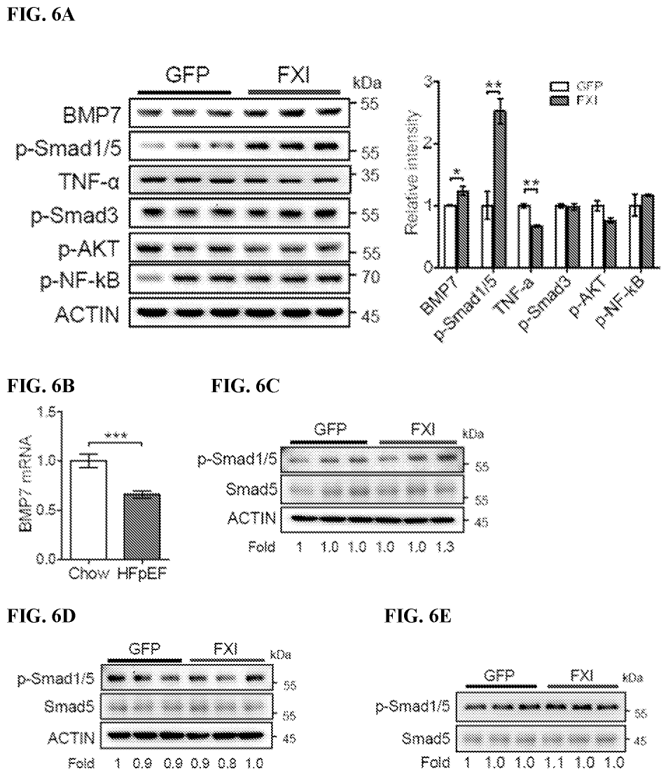

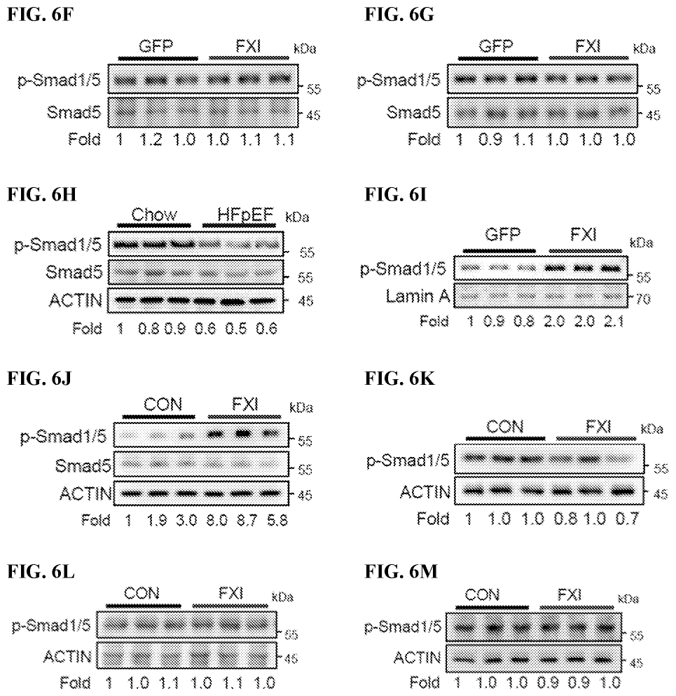

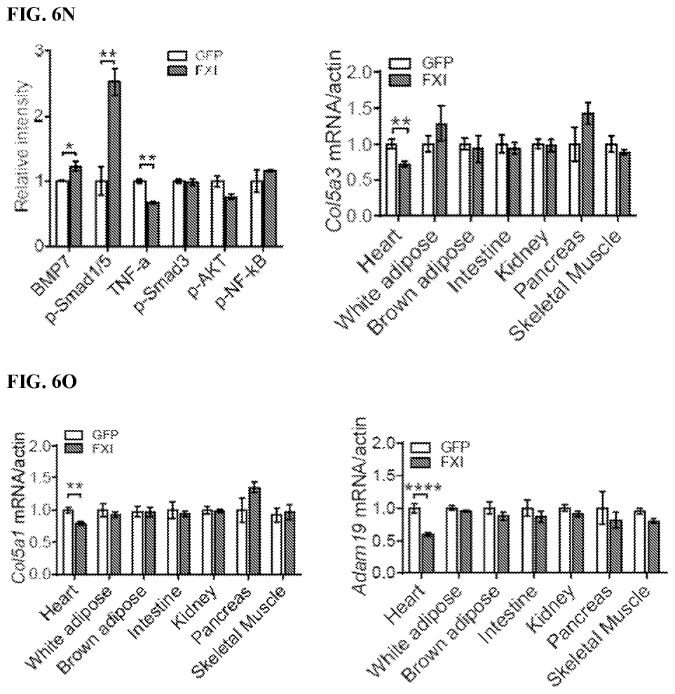

To investigate the molecular mechanism underlying FXI impact on the heart, key pathways predicted from mouse HMDP and human GWAS cohorts were tested. Importantly, FXI induced an increase in BMP7, Smad1/5 phosphorylation, and a decrease in TNF-α in the heart but not in other tissues, indicating activation of the BMP-Smad1/5 pathway and a decrease of inflammation in the heart (FIGS. 6A-6H). To test whether nuclear p-Smad1/5 was also increased, the nuclear fraction was isolated from the same heart tissue. Smad1/5 phosphorylation was significantly induced in the FXI overexpression group relative to GFP control (FIG. 6I). C57BL/6J male mice were injected with control or mouse FXI protein for 2 hours. Phosphorylation of Smad1/5 was observed in the heart but not in other tissues, supporting the tissue-specific activation of the BMP-Smad1/5 pathway (FIGS. 6J-6M). In addition, FXI overexpression reversed the expression of the fibrotic and inflammatory genes Col5a1, Col5a3, and Adam19 in the heart but not in other tissues, consistent with the observed decrease in fibrosis and inflammation in the heart (FIGS. 6N and 60).

### Example 8: FXI Protein Activates the BMP-Smad1/5 Pathway in Cardiomyocytes

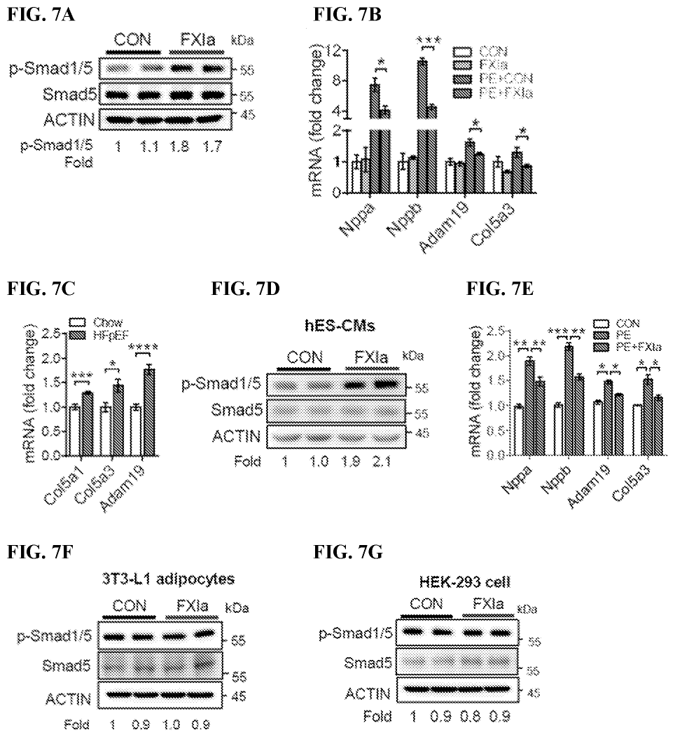

To directly test whether FXI protein activates the BMP-Smad1/5 pathway in cardiomyocytes, neonatal rat ventricular myocytes (NRVMs), human ES induced cardiomyocytes (hES-CMs), and other cell lines were incubated with control media or media containing human activated FXI protein (FXIa) in the presence of phenylephrine (PE) for 24 hours. FXIa increased phosphorylation of Smad1/5 and decreased expression of Nppa, Nppb, Col5a3, and Adam19 in NRVMs and hES-CMs but not in other cell types (FIGS. 7A-7I).

### Example 9: FXI Overexpression Activates BMP Signaling to Protect Against Diastolic Dysfunction

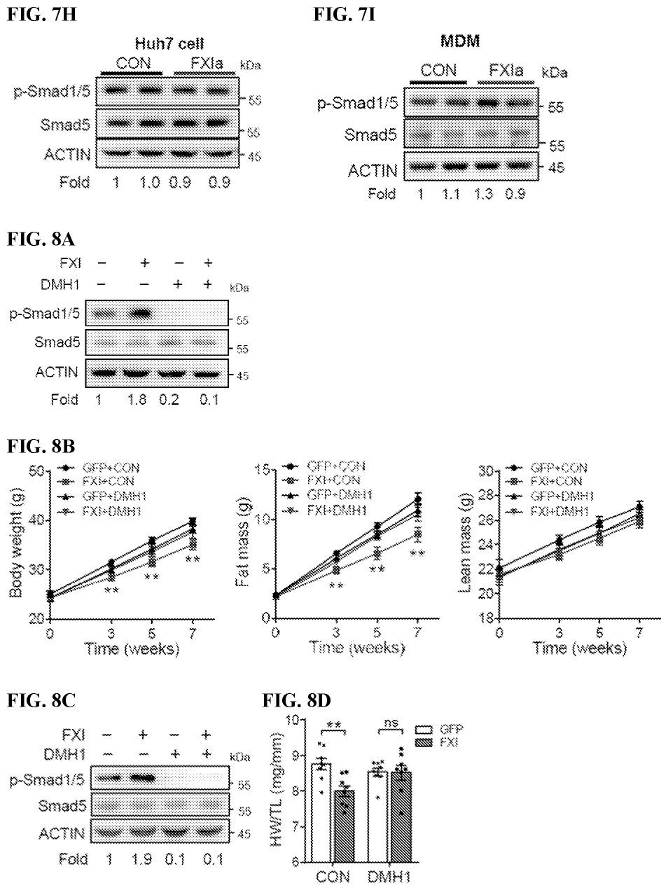

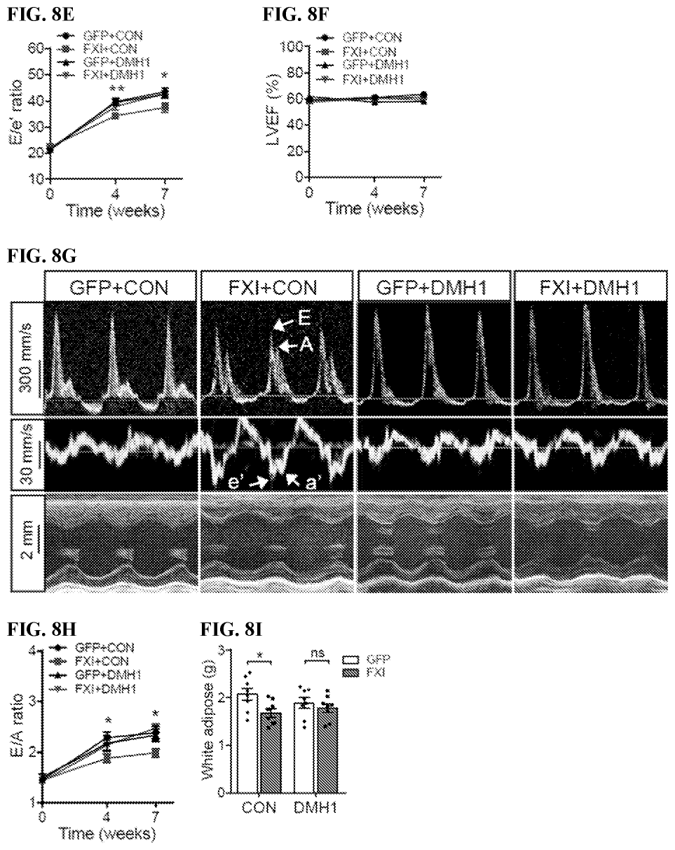

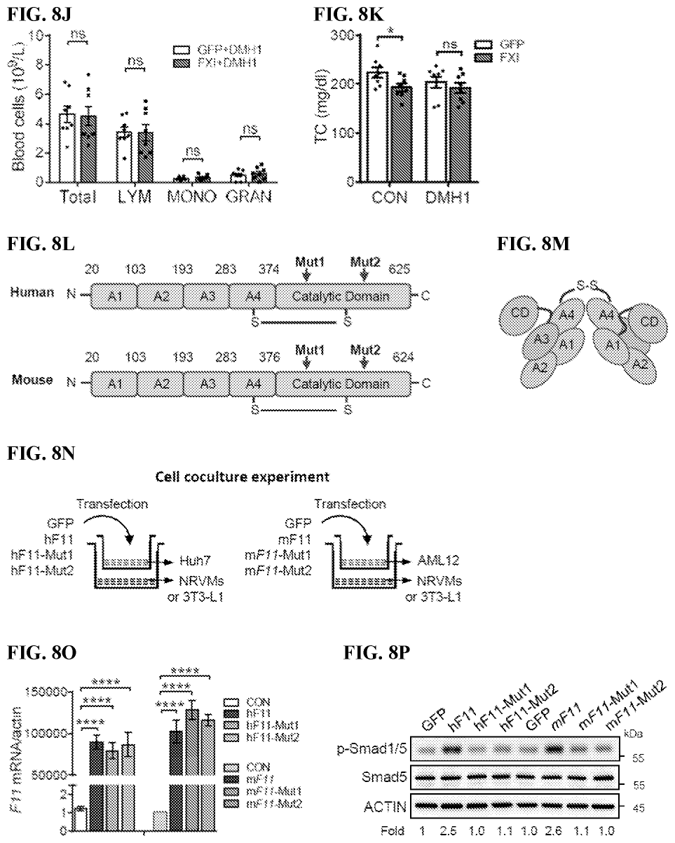

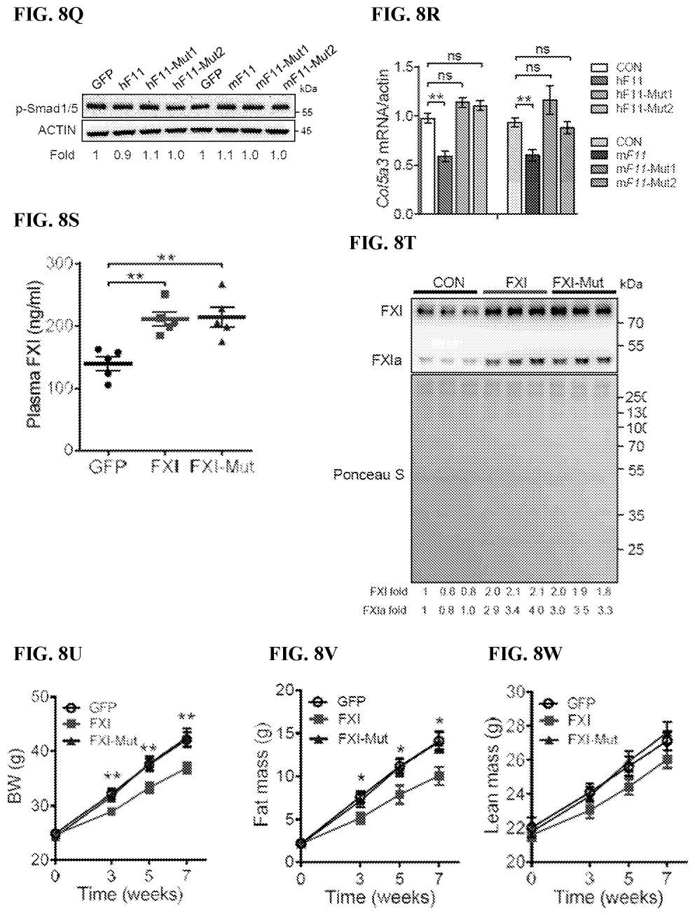

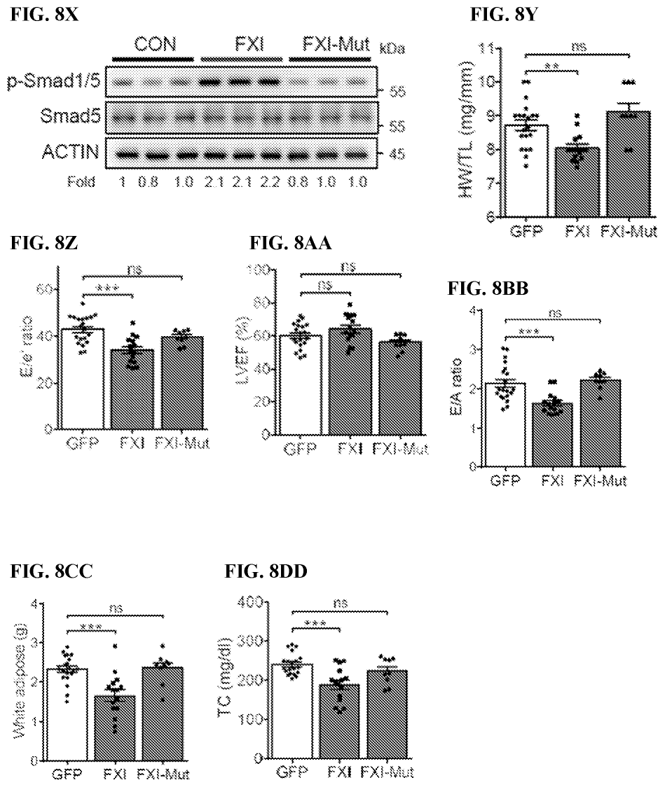

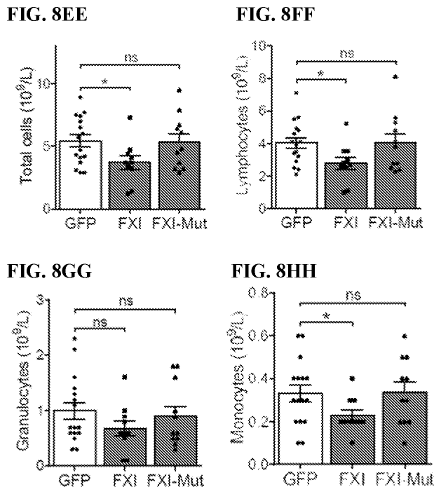

To confirm that FXI overexpression activates BMP signaling to protect against diastolic dysfunction, the BMP type I receptor was blocked with dorsomorphin homolog 1 (DMH1)16. In NRVMs, DMH1 treatment suppressed Smad1/5 phosphorylation induction by FXIa (FIG. 8A). The effects of DMHI administration in vivo was also examined. C57BL/6J mice were injected with AAV8-F11 and fed an HFD+1-NAME for 7 weeks. Injection of DMH1 every other day to block Smad1/5 phosphorylation¹⁷ suppressed the change of body mass induced by FXI (FIG. 8B) as well as the effect of FXI on p-Smad1/5 levels, diastolic function, adipose weight, blood cell numbers, and plasma cholesterol (FIGS. 8C-8K 3E-3H). These results confirmed that FXI protects against HFpEF by activating the BMP pathway.

The FXI protein is conserved in human, mouse, rat, and other species. It consists of four apple domains and one catalytic domain (FIG. 8L). FXI protein is present in plasma as a zymogen, which exists as a homodimer consisting of two identical polypeptide chains linked by disulfide bonds (FIG. 8M).¹⁸ During FXI activation, an internal peptide bond is cleaved by factor XIIa (or XII) in each of the two chains, resulting in activated factor XIa, a seine protease composed of two heavy and two light chains held together by disulfide bonds (FIG. 8B). To test whether the catalytic domain is required for its function on the heart, two missense mutations were introduced in human and mouse FXI catalytic domains, respectively (FIG. 8L and Table 7). These mutations were predicted to be exposed at the surface of the FXI molecule and cause functional defects (Type II mutation).¹⁹ Next, their function was tested in vitro using a co-culture system. Huh7 human liver cells and AML12 mouse liver cells were transfected with respective human or mouse plasmids containing control, wild-type F11 or F11 with mutations. Then, cells were placed in co-cultures with NRVMs and 3T3-L1 adipocytes (FIG. 8N). 24 hours after transfection, FXI was highly induced in both Huh7 cells and AML12 cells (FIG. 8O). In NRVMs, phosphorylation of Smad1/5 was induced by wild-type FXI overexpression from both human and mouse liver cells while mutant FXI did not exhibit a comparable effect (FIG. 8P). In contrast, Smad1/5 phosphorylation was not significantly induced by FXI in 3T3-L1 adipocytes, indicating a heart-specific effect (FIG. 8Q). Consistent with phosphorylated Smad1/5, Col5a3 was decreased by wild-type FXI but not mutant FXI in NRVMs, indicating that the catalytic domain is required for its effect (FIG. 8R).

TABLE 7
Mutated Catalytic Domains

Homo sapiens coagulation factor XI (F11)
(SEQ ID NO: 43
Mutation sites are bolded and enlarged
MIFLYQVVHFILFTSVSGECVTQLLKDTCFEGGDITTVFTPSAKY
CQVVCTYHPRCLLFTFTAESPSEDPTRWFTCVLKDSVTETLPRVN
RTAAISGYSFKQCSHQISACNKDIYVDLDMKGINYNSSVAKSAQE
CQERCTDDVHCHEFTYATRQFPSLEHRNICLLKHTQTGTPTRITK
LDKVVSGFSLKSCALSNLACIRDIFPNTVFADSNIDSVMAPDAFV
CGRICTHHPGCLFFTFFSQEWPKESQRNLCLLKTSESGLPSTRIK
KSKALSGESLQSCRHSIPVFCHSSFYHDTDELGEELDIVAAKSHE
ACQKLCTNAVRCQFFTYTPAQASCNEGKGKCYLKLSSNGSPTKIL
HGRGGISGYTLRLCKMDNECTTKIKPRI [symbol] GGTASVRGEWPWQVTL
HTTSPTQRHLCGGSIIGNQWILTAAHCFYGVESPKILRVYSGILN
QSEIKEDTSFFGVQEIIIHDQYKMAESGYDIALLKLETTVNYTDS
QRPICLPSKGDRNVIYTDCWVTGWGYRKLRDKIQNTLQKAKI [symbol] LV
TNEECQKRYRGHKITHKMICAGYREGGKDACKGDSGGPLSCKHNE
VWHLVGITSWGEGCAQRERPGVYTNVVEYVDWILEKTQAVTRTRP
LEQKLISEEDLAANDILDYKDDDDKV
hF11-Mut1: 389 GTT (Val) → ATT (Ile)
hF11-Mut2: 538 CCC (Pro) → CTC (Leu)
Mus musculus coagulation factor XI (F11)
(SEQ ID NO: 44)
Mutation sites are bolded and enlarged
MTSLHQVLYFIFFASVSSECVTKVFKDISFQGGDLSTVFTPSATY
CRLVCTHHPRCLLFTEMAESSSDDPTKWFACILKDSVTEILPMVN
MTGAISGYSFKQCPQQLSTCSKDVYVNLDMKGMNYNSSVVKNARE
CQERCTDDAHCQFFTYATGYFPSVDHRKMCLLKYTRTGTPTTITK
LNGVVSGFSLKSCGLSNLACIRDIFPNTVLADLNIDSVVAPDAFV
CRRICTHHPTCLEFTFFSQAWPKESQRHLCLLKTSESGLPSTRIT
KSHALSGFSLQHCRHSVPVECHPSFYNDTDFLGEELDIVDVKGQE
TCQKTCTNNARCQFFTYYPSHRLCNERNRRGRCYLKLSSNGSPTR
ILHGRGGISGYSLRLCKMDNVCTTKINPRV [symbol] GGAASVHGEWPWQV
TLHISQGHLCGGSIIGNQWILTAAHCESGIETPKKLRVYGGIVNQ
SEINEGTAFFRVQEMIIHDQYTTAESGYDIALLKLESAMNYTDFQ
RPICLPSKGDRNAVHTECWVTGWGYTALRGEVQSTLQKAKV [symbol] LVS
NEECQTRYRRHKITNKMICAGYKEGGKDTCKGDSGGPLSCKYNGV
WHLVGITSWGEGCGQKERPGVYTNVAKYVDWILEKTQTV
mF11-Mut1: 391 Val (GTA) → Ile (ATA)
mF11-Mut2: 537 Pro (CCA) → Leu (CTA)

To test the effect of missense mutation in vivo, AAV8 with the mouse wild-type and mutant F11 coding sequences were produced. AAV8 containing GFP control, wild-type F11, and mutant F11 (mF11-Mut2) were injected into C57BL/6J male mice and then fed an HFD+1-NAME diet for 7 weeks. After 7 weeks, plasma FXI level was increased in the FXI group and was comparable with the mutant FXI group (FIGS. 3S and 3T). Body weight and fat mass were decreased by wild-type FXI overexpression, but there was no significant difference between the mutant FXI and GFP control groups (FIGS. 8U-8W), indicating functional defects of mutant FXI. Importantly, the effects of FXI on p-Smad1/5, heart weight, E/A ratio, E/e′ ratio, adipose weight, plasma cholesterol, and blood immune cells were not observed in mice carrying mutant FXI, demonstrating that catalytic domain is essential for its function in protecting against deleterious phenotypes in HFpEF (FIG. 3J-3M and S7L-S7R).

### Example 10

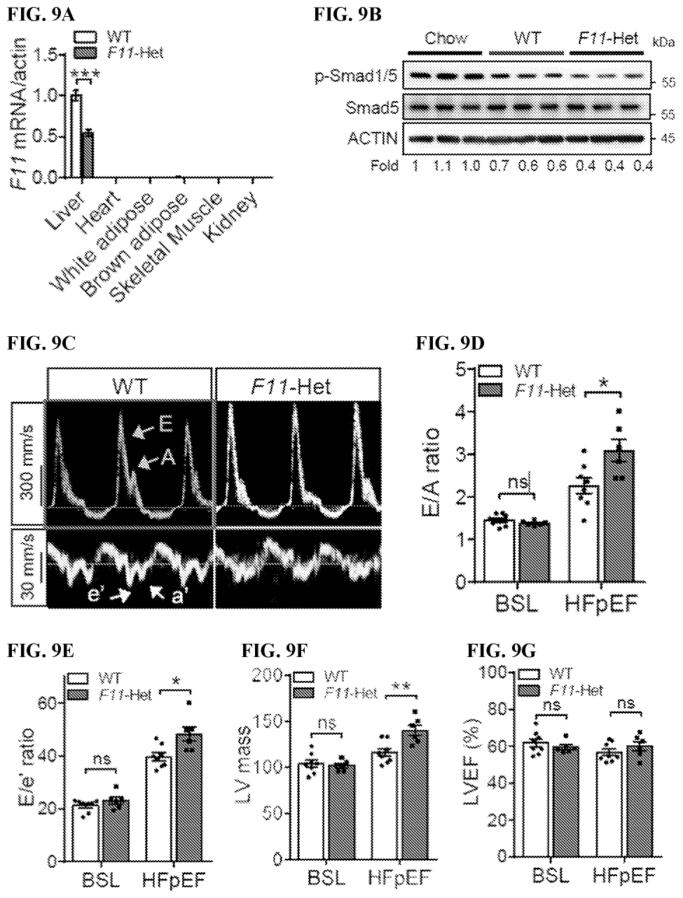

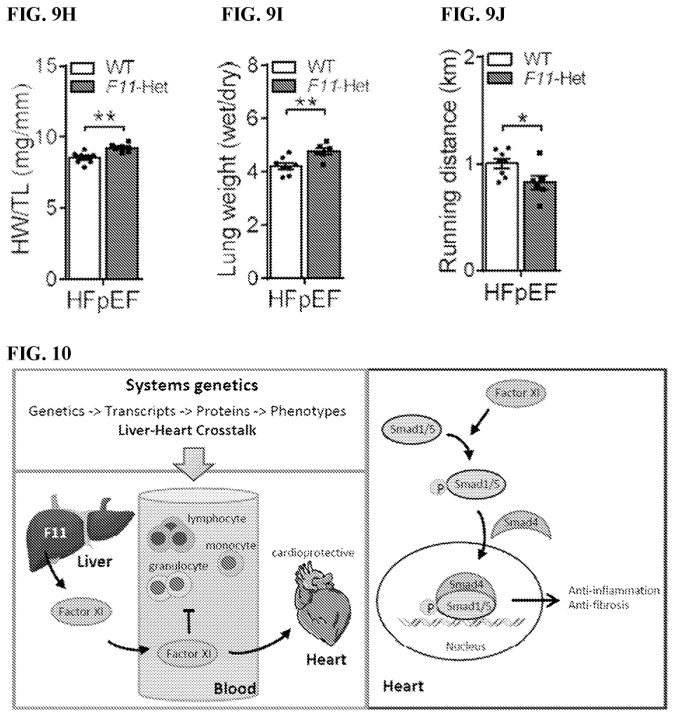

The cardioprotective effect of FXI was further examined using FXI knockout mice in which the F11 gene was disrupted by a PGK-neo cassette.²⁰ FXI transcripts in the liver of heterozygous null mice (F11-Het) were reduced by about 50% compared with wild-type (WT) littermates (FIG. 9A). FXI was either absent or barely detectable in other tissues (FIGS. 2B, 2B, and 9A). Adult WT and F11-Het mice were then fed an HFD+1-NAME diet for 7 weeks to induce HFpEF phenotypes. Compared with WT littermates on the HFpEF diet, p-Smad1/5 was reduced in the hearts of F11-Het mice (FIG. 9B). Consistent with reduced p-Smad1/5 levels, F11-Het mice exhibited more severe diastolic dysfunction, as examined by increased E/A ratio, E/e′ ratio, and LV mass but preserved ejection fraction (FIGS. 9C-9G). Moreover, heart weight and lung weight were higher in F11-Het mice relative to WT controls, suggesting cardiac hypertrophy and lung congestion in FXI deficient mice (FIGS. 9H-91). The exercise tolerance was also decreased in F11-Het mice compared to WT mice (FIG. 9J). These results collectively demonstrated the increased severity of diastolic dysfunction in FXI deficient mice.

The studies presented herein indicate that liver-derived FXI specifically regulates cardiomyocytes through the BMP-Smad1/5 pathway, resulting in attenuation of fibrosis, inflammation, and diastolic dysfunction the context of the HFpEF model (FIG. 10). FXI overexpression also influenced various systemic aspects of metabolism, and the possibility that it may affect organs other than the heart cannot be ruled out. FXI is a component of the intrinsic pathway of blood coagulation, acting downstream of factor XII and functioning as a protease to activate FIX.^{13, 14, 18, 21, 22} FXI-deficient patients generally do not have spontaneous bleeding, as FXI is not required for the initial thrombin generation step,²³ consistent with the possibility that it exhibits other, previously unknown functions. Inactivating mutations of the F11 gene are relatively common among Ashkenazi Jews.²⁴ A number of studies investigated the relationship between FXI and incident coronary heart disease, stroke and ischemic cardiomyopathy.^{25, 26} FXI was reported to inhibit the inflammatory response of Gram-positive pneumonia independent of the intrinsic coagulation activity²⁷. The study described herein indicates that FXI is a direct mediator of liver-heart communication with potential therapeutic applications in heart failure.

### REFERENCES

1. Friedman J M and Halaas J L. Leptin and the regulation of body weight in mammals. Nature. 1998; 395:763-70.
2. Moller S and Bernardi M. Interactions of the heart and the liver. Eur Heart J. 2013; 34:2804-11.
3. Baskin K K, Bookout A L and Olson E N. The heart-liver metabolic axis: defective communication exacerbates disease. EMBO Mol Med. 2014; 6:436-8.
4. Packer M. Atrial Fibrillation and Heart Failure With Preserved Ejection Fraction in Patients With Nonalcoholic Fatty Liver Disease. Am J Med. 2020; 133:170-177.
5. Pacifico L, Di Martino M, De Merulis A, Bezzi M, Osbom J F, Catalano C and Chiesa C. Left ventricular dysfunction in obese children and adolescents with nonalcoholic fatty liver disease. Hepatology. 2014; 59:461-70.
6. Seldin M M, Koplev S, Rajbhandari P, Vergnes L, Rosenberg G M, Meng Y, Pan C, Phuong T M N, Gharakhanian R, Che N, Makinen S, Shih D M, Civelek M, Parks B W, Kim E D, Norheim F, Chella Krishnan K, Hasin-Brumshtein Y, Mehrabian M, Laakso M, Drevon C A, Koistinen H A, Tontonoz P, Reue K, Cantor R M, Bjorkegren J L M and Lusis A J. A Strategy for Discovery of Endocrine Interactions with Application to Whole-Body Metabolism. Cell metabolism. 2018; 27:1138-1155 e6.
7. Lusis A J, Seldin M M, Allayee H, Bennett B J, Civelek M, Davis R C, Eskin E, Farber C R, Hui S, Mehrabian M, Norheim F, Pan C, Parks B, Rau C D, Smith D J, Vallim T, Wang Y and Wang J. The Hybrid Mouse Diversity Panel: a resource for systems genetics analyses of metabolic and cardiovascular traits. J Lipid Res. 2016; 57:925-42.
8. Gandhi P U, Gaggin H K, Redfield M M, Chen H H, Stevens S R, Anstrom K J, Semigran M J, Liu P and Januzzi J L, Jr. Insulin-Like Growth Factor-Binding Protein-7 as a Biomarker of Diastolic Dysfunction and Functional Capacity in Heart Failure With Preserved Ejection Fraction: Results From the RELAX Trial. JACC Heart Fail. 2016; 4:860-869.
9. Dunlay S M, Roger V L and Redfield M M. Epidemiology of heart failure with preserved ejection fraction. Nat Rev Cardiol. 2017; 14:591-602.
10. Hogg K, Swedberg K and McMurray J. Heart failure with preserved left ventricular systolic function; epidemiology, clinical characteristics, and prognosis. J Am Coll Cardiol. 2004; 43:317-27.
11. Owan T E, Hodge D O, Herges R M, Jacobsen S J, Roger V L and Redfield M M. Trends in prevalence and outcome of heart failure with preserved ejection fraction. N Engl J Med. 2006; 355:251-9.
12. Schiattarella G G, Altamirano F, Tong D, French K M, Villalobos E, Kim S Y, Luo X, Jiang N, May H I, Wang Z V, Hill T M, Mammen P P A, Huang J, Lee D I, Hahn V S, Sharma K, Kass D A, Lavandero S, Gillette T G and Hill J A. Nitrosative stress drives heart failure with preserved ejection fraction. Nature. 2019; 568:351-356.
13. Walsh P N. Roles of platelets and factor XI in the initiation of blood coagulation by thrombin. Thromb Haemost. 2001; 86:75-82.
14. Emsley J, McEwan P A and Gailani D. Structure and function of factor XI. Blood. 2010; 115:2569-77.
15. Kandis H, Ozhan H, Ordu S, Erden I, Caglar O, Basar C, Yalcin S, Alemdar R and Aydin M. The prognostic value of mean platelet volume in decompensated heart failure. Emerg Med J. 2011; 28:575-8.
16. Petersen M A, Ryu J K, Chang K J, Etxeberria A, Bardehle S, Mendiola A S, Kamau-Devers W, Fancy SPJ, Thor A, Bushong E A, Baeza-Raja B, Syme C A, Wu M D, Rios Coronado P E, Meyer-Franke A, Yahn S, Pous L, Lee J K, Schachtrup C, Lassmann H, Huang E J, Han M H, Absinta M, Reich D S, Ellisman M H, Rowitch D H, Chan J R and Akassoglou K. Fibrinogen Activates BMP Signaling in Oligodendrocyte Progenitor Cells and Inhibits Remyelination after Vascular Damage. Neuron. 2017; 96:1003-1012 e7.
17. Shan Y, Yang F, Tang Z, Bi C, Sun S, Zhang Y and Liu H. Dexmedetomidine Ameliorates the Neurotoxicity of Sevoflurane on the Immature Brain Through the BMP/SMAD Signaling Pathway. Front Neurosci. 2018; 12:964.
18. Fujikawa K, Chung D W, Hendrickson L E and Davie E W. Amino acid sequence of human factor XI, a blood coagulation factor with four tandem repeats that are highly homologous with plasma prekallikrein. Biochemistry. 1986; 25:2417-24.
19. Saunders R E, Shiltagh N, Gomez K, Mellars G, Cooper C, Perry D J, Tuddenham E G and Perkins S J. Structural analysis of eight novel and 112 previously reported missense mutations in the interactive FXI mutation database reveals new insight on FXI deficiency. Thromb Haemost. 2009; 102:287-301.
20. Gailani D, Lasky N M and Broze G J, Jr. A murine model of factor XI deficiency. Blood Coagul Fibrinolysis. 1997; 8:134-44.
21. Wu W, Sinha D, Shikov S, Yip C K, Walz T, Billings P C, Lear J D and Walsh P N. Factor XI homodimer structure is essential for normal proteolytic activation by factor XIIa, thrombin, and factor XIa. J Biol Chem. 2008; 283:18655-64.
22. Bolton-Maggs P H. Factor XI deficiency. Baillieres Clin Haematol. 1996; 9:355-68.
23. Wheeler A P and Gailani D. Why factor XI deficiency is a clinical concern. Expert Rev Hematol. 2016; 9:629-37.
24. Asakai R, Chung D W, Davie E W and Seligsohn U. Factor XI deficiency in Ashkenazi Jews in Israel. N Engl J Med. 1991; 325:153-8.
25. Appiah D, Fashanu O E, Heckbert S R, Cushman M, Psaty B M and Folsom A R. Relation of coagulation factor XI with incident coronary heart disease and stroke: the Cardiovascular Health Study. Blood Coagul Fibrinolysis. 2017; 28:389-392.
26. Zabczyk M, Butenas S, Palka I, Nessler J and Undas A. Active tissue factor and activated factor XI in circulating blood of patients with systolic heart failure due to ischemic cardiomyopathy. Pol Arch Med Wewn. 2010; 120:334-40.
27. Stroo I, Zeerleder S, Ding C, Luken B M, Roelofs J, de Boer O J, Meijers J C M, Castellino F J, van't Veer C and van der Poll T. Coagulation factor XI improves host defence during murine pneumonia-derived sepsis independent of factor XII activation. Thromb Haemost. 2017; 117:1601-1614.
28. Bennett B J, Farber C R, Orozco L, Kang H M, Ghazalpour A, Siemers N, Neubauer M, Neuhaus I, Yordanova R, Guan B, Truong A, Yang W P, He A, Kayne P, Gargalovic P, Kirchgessner T, Pan C, Castellani L W, Kostem E, Furlotte N, Drake T A, Eskin E and Lusis A J. A high-resolution association mapping panel for the dissection of complex traits in mice. Genome Res. 2010; 20:281-90.
29. Wang Y, Huang S, Sah V P, Ross J, Jr., Brown J H, Han J and Chien K R. Cardiac muscle cell hypertrophy and apoptosis induced by distinct members of the p38 mitogen-activated protein kinase family. J Biol Chem. 1998; 273:2161-8.
30. Sharma A, Li G, Rajarajan K, Hamaguchi R, Burridge P W and Wu S M. Derivation of highly purified cardiomyocytes from human induced pluripotent stem cells using small molecule-modulated differentiation and subsequent glucose starvation. J Vis Exp. 2015.
31. Lippert C, Listgarten J, Liu Y, Kadie C M, Davidson R I and Heckerman D. FaST linear mixed models for genome-wide association studies. Nat Methods. 2011; 8:833-5.
32. Langfelder P and Horvath S. WGCNA: an R package for weighted correlation network analysis. BMC Bioinformatics. 2008; 9:559.

### INCORPORATION BY REFERENCE

All publications and patents mentioned herein are hereby incorporated by reference in their entirety as if each individual publication or patent was specifically and individually indicated to be incorporated by reference. In case of conflict, the present application, including any definitions herein, will control.

### EQUIVALENTS

While specific embodiments of the subject invention have been discussed, the above specification is illustrative and not restrictive. Many variations of the invention will become apparent to those skilled in the art upon review of this specification and the claims below. The full scope of the invention should be determined by reference to the claims, along with their full scope of equivalents, and the specification, along with such variations.

1. A method of treating or preventing heart failure with preserved ejection fraction (HfpEF) in a subject, the method comprising administering an FXI polypeptide or fragment thereof or a nucleic acid molecule comprising a sequence encoding an FXI polypeptide or fragment thereof.

2-4. (canceled)

5. The method of claim 1, wherein the FXI polypeptide or fragment thereof comprises a catalytic domain.

6. The method of claim 1, wherein the FXI polypeptide comprises an amino acid sequence having at least 80% sequence identity to SEQ ID NO: 43 or 44.

7. The method of claim 1, wherein the administering comprises pulmonary, rectal, intravenous, subcutaneous, intraperitoneal, oral, intra-arterial, intramuscular, intraventricular, or intranasal administration.

8. The method of claim 1, wherein the FXI polypeptide or fragment thereof or a nucleic acid encoding an FXI polypeptide or fragment thereof is administered one, two, three, or four times a day.

9-10. (canceled)

11. The method of claim 1, wherein the nucleic acid molecule comprises a regulatory sequence operatively linked to the nucleic acid sequence encoding the FXI polypeptide or fragment thereof.

12. (canceled)

13. The method of claim 11, wherein the regulatory sequence is a liver specific promoter.

14. The method of claim 1, wherein the nucleic acid molecule is an adenoviral expression vector.

15-18. (canceled)

19. The method of claim 1, wherein the method comprises administering an additional agent, and the additional agent is a liver-derived hepatocyte growth factor activator (HGFAC) polypeptide or fragment thereof or a nucleic acid molecule encoding an HGFAC polypeptide or fragment thereof, a complement C8 gamma chain (C8G) polypeptide or fragment thereof or a nucleic acid molecule encoding a C8G polypeptide or fragment thereof, phenylephrine, or dorsomorphin homolog 1.

20. (canceled)

21. The method of claim 1, wherein the administering of the FXI polypeptide or fragment thereof or the polynucleotide encoding an FXI polypeptide or fragment thereof modulates at least one of the expression or activity of p-Smad1/5.

22. (canceled)

23. The method of claim 1, wherein the administering attenuates at least one of fibrosis, inflammation, or diastolic dysfunction in heart tissue.

24. A method for activating the BMP-Smad1/5 pathway in heart tissue, the method comprising overexpressing an FXI polypeptide or fragment thereof in a liver cell.

25. The method of claim 24, wherein the FXI polypeptide or fragment thereof comprises a catalytic domain.

26. The method of claim 24, wherein the method further comprises contacting the liver cell with a nucleic acid molecule encoding an FXI polypeptide or fragment thereof.

27. The method of claim 24, wherein the FXI polypeptide comprises an amino acid sequence having at least 80% sequence identity to SEQ ID NO: 43 or 44.

28. The method of claim 24, wherein the contacting comprises transfection, transduction, electroporation, or lipofection.

29-30. (canceled)

31. The method of claim 26, wherein the nucleic acid molecule is DNA and further comprises a regulatory sequence operatively linked to the nucleic acid sequence encoding the FXI polypeptide or fragment thereof.

32. (canceled)

33. The method of claim 31, wherein the regulatory sequence is a liver specific promoter.

34. The method of claim 26, wherein the nucleic acid molecule is an adenoviral expression vector.

35-37. (canceled)

38. A method of monitoring the effectiveness of administering a composition comprising an FXI polypeptide or a nucleic acid molecule encoding an FXI polypeptide or fragment thereof for the treatment of a subject having or suspected of having heart failure with preserved ejection fraction (HFpEF), the method comprising:
performing a first echocardiogram on the subject prior to administering a therapeutically effective amount of the composition comprising an FXI polypeptide or a nucleic acid molecule encoding an FXI polypeptide or fragment thereof,
administering to the subject the composition comprising an FXI polypeptide or a nucleic acid molecule encoding an FXI polypeptide or fragment thereof,
performing at least one additional echocardiogram after administering the composition comprising an FXI polypeptide or a nucleic acid molecule encoding an FXI polypeptide or fragment thereof;
comparing the first echocardiogram to the at least one additional cardiogram, wherein an improved echocardiogram post administration relative to the first echocardiogram is indicative of effectiveness.
